(19) United States
(12) Patent Application Publication
(10) Pub. No.: US 2023/0233532 A1
(43) Pub. Date: Jul. 27, 2023

(54) SPHINGOSINE-1-PHOSPHATE RECEPTOR 1 AGONIST AND LIPOSOMAL FORMULATIONS THEREOF

(71) Applicant: The Penn State Research Foundation, University Park, PA (US)

(72) Inventors: Gavin Peter ROBERTSON, Hummelstown, PA (US); Shantu AMIN, Union City, NJ (US); Pingnain HE, Harrisburg, PA (US); Arun SHARMA, Hummelstown, PA (US); Yu-Chi CHEN, Hershey, PA (US)

(21) Appl. No.: 18/010,690

(22) PCT Filed: Apr. 26, 2021
(86) PCT No.: PCT/US2021/029144

§ 371 Date: Dec. 15, 2022

Related U.S. Application Data
(60) Provisional application No. 63/044,515, filed on Jun. 26, 2020.

(51) Int. Cl. A61K 31/4245; A61K 9/127; A61K 9/16; A61P 35/00

(52) U.S. Cl. CPC A61K 31/4245; A61K 9/1271; A61K 9/1617; A61P 35/00

(57) ABSTRACT

The present disclosure is directed to a compound or compositions comprising the same for decreasing vascular permeability and the prevention or inhibition of metastasis in a cancer.

### CROSS-REFERENCE TO RELATED APPLICATIONS

This application claims the benefit of priority to U.S. Provisional Application No. 63/044,515, filed Jun. 26, 2020, the disclosure of which is incorporated herein by reference in its entirety.

### STATEMENT REGARDING FEDERALLY SPONSORED RESEARCH OR DEVELOPMENT

This invention was made with government support under Grant Nos. CA136667, CA138634, DK097391, and HL130363 awarded by the National Institutes of Health. The Government has certain rights in the invention.

### TECHNICAL FIELD

This disclosure relates to compounds for the prevention or inhibition of metastasis in cancer, and more particularly to sphingosine-1-phosphate receptor 1 agonists and compositions thereof.

### BACKGROUND

Metastasis is the leading cause of cancer morbidity as well as mortality and is responsible for approximately 90% of cancer death (1-3). Metastasis is a general term to describe cancer cells traveling from primary sites to neighboring tissue or distant sites, which involves multiple steps including local invasion, intravasation, survival in circulation, extravasation, and proliferation in distant tissues (4). Each step can be rate-limiting since disruption of any of the multiple processes can retard metastasis (5).

The endothelial cell (EC) layer of vessels plays important roles during intravasation and extravasation in the metastatic cascade (6). While quiescent, non-activated ECs can reduce tumor cell transmigration and metastasis, inflammation increases these processes to promote cancer cell metastasis (7-9). The literature shows that melanomas and circulating cells secrete multiple factors, including thrombin, VEGF and monocyte chemoattractant protein 1 (MCP1/CCL2), which can disrupt endothelial integrity by triggering actin remodeling, cellular contractility and junctional disassembly (10-14).

Increases in vascular permeability mediated by inflammatory mediators can cause changes in EC junctions through modifications in adherens and tight junctions (15). Vascular endothelial (VE) cadherin at the edges of ECs plays a major role intracellularly linking actin filament in the cytoskeleton (16). Inflammatory factors can cause internalization of VE-cadherin, whose absence then disrupts the cellular interactions leading to gap formation. In addition, contractile forces mediated by actin and myosin further promotes gap formation (17).

The actin-myosin action depends on the phosphorylation status of myosin light chain (MLC) (18). Increased phosphorylated-MLC (pMLC) levels triggered by inflammatory mediators causes changes in the actin stress fiber leading to the formation of gaps in the EC layer (19).

Sphingosine-1-phosphate (S1P) is a natural lipid, and its levels in the plasma has been identified as a biomarker for vascular barrier integrity (20). Cell culture studies showed that S1P increased the levels of VE-cadherin and p-catenin at EC junctions and enhanced adherens and tight junctional assembly (21). Studies in intact microvessels revealed that the effect of S1P on maintaining microvessel permeability occurred through the activation of EC S1PR1, and the blockage of S1PR1 abolished the inhibitory effect of S1P on inflammatory mediator-induced increases in microvessel permeability (22). This disclosure translates these observations to cancer treatment by developing a bioavailable S1PR1 agonists to enhance the endothelium barrier integrity and reverse the effects of inflammatory mediators, thereby inhibiting the transit of metastatic cells across vascular walls.

Several S1P receptor agonists have been developed for treating inflammatory diseases but none are useful for clinical cancer therapy (23). The most well-known S1P analog, FTY720 (fingolimod) is FDA-approved for treating multiple sclerosis but is not useful in cancer because it acts as a broad spectrum S1PR1/3/4/5 agonist (24). This leads to S1PR1 degradation and lymphopenia making it unsuitable to strengthen the endothelial barrier and reduce metastasis (25). SEW2871, a S1PR1-selective agonist, has been shown to fully replicate the action of S1P on endothelium integrity in intact microvessels, and potently inhibited the inflammatory mediator-induced increases in vascular permeability (22). Unfortunately, SEW2871 is not bioavailable requiring solubilization in a DMSO containing solution, which limits its clinical utility, but is useful for research purposes. The chemical structure of SEW2871 also prevents it from being incorporated into a bioavailable nanoliposomal formulation.

There is a clear need for therapeutics that can be used in the treatment and prevention of metastasis.

### SUMMARY

The present disclosure provides a compound, or a pharmaceutically acceptable salt, prodrug, or derivative thereof, that attenuates the metastasis of cancers in subjects in need thereof by selectively activating sphingosine-1-phosphate receptor 1 (S1PR1) to tighten the junctions between endothelial cells in the vascular endothelium. The presently disclosed compound can also more readily formulated as a liposome as compared to other S1PR1 agonists, allowing it to be more readily delivered to desired vascular tissue.

Thus in one aspect, Compound I is provided of the following formula

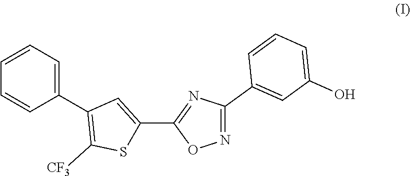 [SMILES: Oc1cccc(-c2noc(-c3cc(-c4ccccc4)c(C(F)(F)F)s3)n2)c1]

or a pharmaceutically acceptable salt, prodrug, or derivative thereof.

A pharmaceutical composition is also provided comprising Compound I, or a pharmaceutically acceptable salt, prodrug, or derivative thereof, and a pharmaceutically acceptable carrier.

The present disclosure also provides a formulation comprising:

a plurality of nanoliposomes comprising a phospholipid and a PEG-phospholipid; and

Compound I, or a pharmaceutically acceptable salt, prodrug, or derivative thereof, encapsulated within the plurality of nanoliposomes.

In some embodiments, the phospholipid comprises L-α-phosphatidylcholine (ePC). In some embodiments, the PEG-phospholipid comprises 1,2-dipalmitoyl-sn-glycero-3-phosphoethanolamine-N-[methoxy(polyethylene glycol)-2000] ammonium salt (DPPE-PEG 2000).

In another aspect, a method of treating or preventing metastasis of a cancer in a subject in need thereof is provided, the method comprising administering a therapeutically effective amount of Compound I, or a pharmaceutically acceptable salt, prodrug, or derivative thereof.

In another aspect, a method of treating or preventing diabetes, hyperglycemia, or complications arising therefrom in a subject in need thereof is provided, the method comprising administering a therapeutically effective amount of Compound I, or a pharmaceutically acceptable salt, prodrug, or derivative thereof.

In another aspect, a method of decreasing vascular permeability in a subject in need thereof is provided, the method comprising administering a therapeutically effective amount of Compound I, or a pharmaceutically acceptable salt, prodrug, or derivative thereof.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIGS. 1A-1C show the development of S1PR1 agonist Compound I (ASR396). FIG. 1A shows docking studies that were performed on the active site pocket of S1PR1 to evaluate the binding of SEW2871 and Compound I (ASR396) compounds. FIG. 1B shows a representative synthetic scheme for Compound I (ASR396). FIG. 1C shows binding affinity of SEW2871 and Compound I (ASR396) to S1PR1.

FIGS. 2A-2G show characterization of the nanoliposomal formulation of Compound I (nanoASR396). The nanoliposomal formulation was characterized by: (FIG. 2A) Cryogenic electron microscopy. The size of particles ranged from 10 nm to 80 nm in diameter. Scale bar=100 nm; (FIG. 2B) drug encapsulation; (FIG. 2C) release kinetics; and (FIGS. 2D and 2E) stability by examination of size and charge changes over a 6-week period. Drug encapsulation was calculated by estimating the concentration of Compound I (ASR396) in the nanoliposomes before and after membrane filtration. In-vitro release kinetics were performed using dialysis in a saline solution. (FIG. 2F) A 15-days of continuous daily treatment with Compound I (ASR396) nanoliposomes toxicity evaluation demonstrated no toxicity as evidenced through behavioral or weight changes in animals. Treatment did not lead to animal mortality. (FIG. 2G) Assessment of serum biomarkers of major organ functions to determine toxicity, showed no different between drug compared to vehicle treated animal controls following 15-days of continuous daily treatment with Compound I (ASR396) nanoliposomes. Empty nanoliposomes (Lipo) served as the control to which Compound I (ASR396) was compared.

FIGS. 3A and 3B show activation of S1PR1 with agonist SEW2871 significantly attenuated platelet activating factor (PAF)-induced increases in rat microvessel permeability. Microvessel permeability was assessed by measuring one of the permeability coefficients hydraulic conductivity, L_{P}, in individually perfused intact rat mesenteric venules. (FIG. 3A) An individual representative experiment shows the time course of the L_{P} changes measured in the same vessel. (FIG. 3B) Summary results of 6 experiments. Each vessel had control and all treatment measurements as that shown in FIG. 3A, and the L_{P} changes were compared with its own control. Perfusion of vessels with SEW2871 (SEW, 10 μM) did not alter baseline L_{P} increase, but significantly inhibited PAF-induced Lₚ increase. After washing away SEW2871 and PAF with BSA-Ringer perfusion for 40 min, each vessel was exposed to a second application of PAF and showed a typical PAF-induced L_{P} increase, indication each vessel was responsive to PAF. ns p≥0.23. ****p≤0.0001.

FIGS. 4A-4C show that the nanoliposomal formulation of Compound I (nanoASR396) inhibits PAF-induced permeability increases in intact rat microvessels by activation of endothelial S1PR1. (FIGS. 4A and 4C) Hydraulic conductivity (L_{P}) measured in individually perfused intact rat microvessels showed that the application of the nanoliposomal formulation (nanoASR, 100 μM) did not alter baseline L_{P} but significantly attenuated a PAF (10 nM)-induced L_{P} increase. The perfusion of empty nanoliposome (Lipo) alone showed no such effect. The time courses of the L_{P} changes from two individual experiments using the nanoliposomal formulation (nanoASR396) and empty nanoliposome were overlaid in (FIG. 4A) to show the contrasting effects on PAF-induced L_{P} increases. When each vessel was exposed to PAF alone for the second time, both vessels responded to PAF to a similar magnitude, indicating the suppressed L_{P} response to PAF in the presence of the nanoliposomal formulation was due to the inhibitor effect of released ASR396, not variability in vessel response. (FIGS. 4B and 4C) The nanoliposomal formulation effect on PAF-induced L_{P} increase was due to S1PR1 activation on the endothelium. Pre-perfusing vessels with 10 μM W146, an antagonist of S1PR1, abolished the inhibitory effect of the nanoliposomal formulation on PAF-induced L_{P} increase. (FIG. 4B) The overlaid two individual experiments demonstrated the contrast of PAF-induced L_{P} increase. (FIG. 4C) Results summary. ns p≥0.71 *p=0.038. **p=0.002. ***p=0.001. ****p≤0.001.

FIGS. 5A-5K show that the nanoliposomal formulation of Compound I (nanoASR396) reverses the effects of inflammatory mediators on the cultured endothelial cell monolayer model. (FIG. 5A) Representative images showed the VE-cadherin distribution after Compound I (ASR396), VEGF, BK, IL-8 and the combination of Compound I plus the inflammatory mediator treatment on ECs in the monolayer EC model. Arrows point to the discontinuous VE-cadherin line at the EC membrane junction, indicating gap formation. Scale bar: 20 μm. (FIGS. 5B, 5C, and 5D) 5 μM SEW2871 and Compound I (ASR396) reduced the EC gap formation induced by: (FIG. 5B) 100 ng/ml VEGF, (FIG. 5C) 0.5 μM BK and (FIG. 5D) 150 ng/ml IL-8. SEW2871 and Compound I alone did not affect EC gap formation. (FIG. 5E) 5 μM W146 S1PR1 antagonist blocked the effect of Compound I (ASR396) on BK-mediated EC gap formation, demonstrating ASR396 binding to S1PR1. (FIG. 5F) Compound I (ASR396) and SEW2871 promoted S1PR1 internalization in HEK293A-S1P1-GFP cells, which was inhibited by S1PR1 antagonist W146. (FIG. 5G) The quantification of S1PR1 internalization following Compound I (ASR396) and SEW2871 treatment of HEK293A-S1P1-GFP cells. Internalization was inhibited by S1PR1 antagonist W146. (FIG. 5H) Inflammatory mediators and Compound I (ASR396) modulated phosphorylation of MLC, BK, VEGF and IL-8 increased the pMLC levels on wester blots, while Compound I reduced the VEGF- and IL-8-triggered MLC phosphorylation. (FIGS. 5I, 5J, and 5K) Modulation of EC layer gap formation by the nanoliposomal formulation of Compound I (nanoASR396). The nanoliposomal formulation inhibited (FIG. 5I) VEGF-, (FIG. 5J) BK- and (FIG. 5K) IL-8-induced gap formation.

FIGS. 6A-6E show that the nanoliposomal formulation of Compound I (nanoASR396) inhibited melanoma lung metastasis. (FIG. 6A) Overview of the treatments protocol to inhibit experimental melanoma cell metastasis. (FIG. 6B) 6, 12, or 18 hours prior to A375M-GFP melanoma cell injection, animals were treated with 15 mg/kg of the nanoliposomal formulation of Compound I (nanoASR396) and 29 days later GFP-tagged metastatic lung nodules quantified. Left: representative images of lungs under each treatment. Right: metastatic nodule quantification. (FIGS. 6C and 6D) 6 hours prior to UACC 903M-GFP and 1205 Lu-GFP melanoma cell injections, animals were treated with 15 mg/kg of the nanoliposomal formulation and 29 days later GFP-tagged metastatic lung nodules quantified. Left: representative images of lungs under each treatment. Right: metastatic nodule quantification. (FIG. 6E) 6, 12 or 18 hours prior to A375M-GFP melanoma cell injection, animals were treated with control intraperitoneally administered 5 mg/kg SEW2871 and 29 days later GFP-tagged metastatic lung nodules quantified. Left: representative images of lungs under each treatment. Right: metastatic nodule quantification.

FIGS. 7A-7C show measurements of permeability coefficients, hydraulic conductivity (L_{P}) in individually perfused rat mesenteric microvessels. FIG. 7A. A top view of the experimental setup. A single mesenteric microvessel from an anesthetized rat was cannulated with a perfusion pipette and the surrounding circulation remains intact. FIG. 7B. Images of a cannulated venular microvessel in the rat mesentery. FIG. 7C. Schematic illustration of the measurements of Lp. The cannulation pipette is connected to a monometer that applies perfusion pressure for the perfusate. A small amount of rat RBCs (1%) is added to the perfusate as marker cells. The vessel is briefly occluded downstream during each measurement and the velocity of the marker cell movement (Vcell) during the vessel occlusion period was used to calculate water flux (Jv). The parameters used for Lp calculation are illustrated, which are based on the Starling relations for fluid ultrafiltration. In each experiment, a vessel was cannulated and perfused with albumin-Ringer's solution (10 mg BSA/ml) first to measure baseline Lp. Then the same vessel was recannulated with a micropipette containing different test solutions to measure the changes in Lp (relative to its own baseline control).

FIG. 8 shows that screening of inflammatory inhibitors showed that S1PR1 agonists (SEW2871/Compound I i.e., ASR396) inhibited EC gap formation while other clinically available inflammatory inhibitors did not decrease EC gap formation. SEW2871/ASR296: S1PR1 agonist, Nintedanib: VEGFR inhibitor, A286982: ICAM-1 inhibitor, Roflumilast: PDE-4 inhibitor, Tacrolimus: mTOR inhibitor.

FIGS. 9A-9C show Erebus predicted binding scaffolds (shown in gray dots) from (FIG. 9A) 3V2Y and (FIG. 9B) 3WKE superposed with the input query structure (shown in cyan and red mesh representation). The co-crystallized ligand bound to S1PR1 in PDB: 3V2Y is shown in van der Waals representation. (FIG. 9C) Molecular docking of ASR396 to the predicted binding scaffold of 3WKE. ASR396 is shown in van der Waals representation.

FIG. 10 shows that Compound I (ASR396) caused no off-target effects on major signaling pathways in HUVEC cells. The phosphorylated protein levels were normalized to total protein levels and the Compound I-treated phosphorylated protein levels were compared to the control (DMSO)-treated ones.

FIGS. 11A-11C show that macrophage inhibitory cytokine 1, MIC-1, was elevated in melanoma. (FIG. 11A) The levels of MIC-1 were higher in the serum of melanoma patients. MIC-1 was measured by ELISA. (FIG. 11B) MIC-1 was expressed in most of the melanoma cell lines, while normal cells, such as fibroblasts, keratinocytes, and melanocytes, did not express MIC-1. (FIG. 11C) The levels of MIC-1 were significantly higher in metastatic melanoma cell lines, A375M and UACC 903M, compared to the non-metastatic melanoma cell line, UACC 903.

FIGS. 12A-12D show that MIC-1 did not affect the proliferation and migration of melanoma cells. (FIG. 12A) A375MshMIC-1 and (FIG. 12B) UACC 903MshMIC-1 cells were created with shRNA plasmids to reduce the levels of p53 in cells. The MIC-1 secretion was significantly reduced in the shMIC-1 cell lines. (FIG. 12C) The cell viability and proliferation were determined by the MTS assay. The reduction in MIC-1 expression did not affect melanoma cell growth. (FIG. 12D) The cell migration was determined by the wound healing assay. There was not difference in cell migration between UACC 903M and UACC 903MshMIC-1 cells.

FIGS. 13A-13D show that MIC-1 synergized with the inflammation to increase endothelial gap and permeability. (FIGS. 13A and 13B) A confluent monolayer of human umbilical vein endothelial cells (HUVEC) was treated with MIC-1 and inflammatory mediators, (FIG. 13A) bradykinin (BK) and (FIG. 13B) VEGF at various concentrations. The endothelial gap formation was determined by staining the VE-cadherin and observing the gaps between HUVEC cells under the fluorescent microscope. MIC-1 alone did not increase the endothelial gap formation but it significantly increased the gap formation induced by BK and VEGF. (FIG. 13C) The endothelial permeability was examined in vivo using the Miles assay. MIC-1 significantly enhanced the endothelial permeability induced by the inflammatory mediator, PAF. (FIG. 13D) MIC-1 dose-dependently increased the myosin light chain (MLC) phosphorylation in HUVEC cells to increase the endothelial gap formation.

FIGS. 14A-14E show that MIC-1 enhanced melanoma cell growth in metastatic lungs. A375M and A375MshMIC-1 cells were injected intravenously into nude mice and allowed to develop lung metastases for 29 days. At the end of experiment, lungs were removed and imaged. Metastatic tumors and dormant cells were counted manually. (FIG. 14A) Representative pictures of lungs with metastases caused by A375M and A375MshMIC-1 cells. Arrowheads indicated dormant cells, and arrows indicated metastatic tumors. (FIG. 14B) Metastases caused by A375MshMIC-1 cells occupied less areas in lungs. (FIG. 14C) More dormant cells were present in lungs of A375MshMIC-1-bearing mice. (FIGS. 14D and 14E) The numbers of tumors and overall metastases did not significantly differ in lungs bearing A375M and A375MshMIC-1 cells.

FIGS. 15A-15E show that Sphingosine-1-phosphate (SIP) agonist, Compound I (ASR396), and its nanoliposomal formation (ASR-Lipo) reduced the endothelial gap formation and permeability induced by MIC-1 and inflammatory mediators. In a confluent HUVEC monolayer model, SIP agonist, (FIG. 15A) SEW2871, (FIG. 15B) Compound I (ASR396), and (FIG. 15C) ASR-Lipo, inhibited the endothelial gap formation induced by MIC-1 and inflammatory mediator, BK. (FIG. 15D) SEW2871 inhibited the endothelial permeability increased by MIC-1 and inflammatory mediator, PAF. (FIG. 15E) Compound I (ASR396) inhibited the MLC phosphorylation induced by MIC-1 and inflammatory mediators.

FIGS. 16A-16C show that SIP agonists reduced melanoma lung metastasis. Nude mice were given 5 mg/kg SEW2871 intraperitoneally or 15 mg/kg ASR-Lipo intravenously 6, 12 and 18 hours before receiving A375M and UACC 903M cells through the tail vein. Lungs were removed and imaged after 29 days. Lung metastases were counted manually. (FIG. 16A) SEW287, used as a positive control, reduced melanoma metastasis. (FIGS. 16B and 16C) ASR-Lipo significantly reduced the numbers of metastatic tumors in (FIG. 16B) A375M- and (FIG. 16C) UAC 903M-bearing mice.

FIGS. 17A-17C show that MIC-1 synergized with PAF to increase endothelial gap formation and microvessel permeability in vivo. The effect of MIC-1 and PAF on microvessel permeability was examined in rat microvessels in vivo. Permeability was assessed by measuring hydraulic conductivity, Lₚ. MIC-1 significantly enhanced the permeability increase induced by PAF. (FIG. 17A) An individual representative experiment shows the time course of the Lp changes when the same vessel was exposed to MIC-1 and MIC-1 plus PAF sequentially. (FIG. 17B) Summary results showing over 3-fold Lₚ increase when PAF was applied to MIC-1 treated vessels compared to vessels exposed to PAF alone. (FIG. 17C) VE-cadherin staining in individually perfused rat mesenteric microvessels (the same type of vessels used for Lₚ measurements) under control, PAF exposure alone and PAF applied to MIC-1 treated conditions. PAF exposure induced redistribution of VE-cadherin showing as discontinued outline at junctions between endothelial cells. More prominent discontinuation of VE-cadherin occurred at endothelial cell junctions when PAF was applied to MIC-1 treated microvessels than that in vessels exposed to PAF alone.

FIG. 18A shows that Compound I (ASR396) inhibited the endothelial gap formation induced by MIC-1 and VEGF in a confluent HUVEC monolayer model. FIG. 18B shows that the nanoliposomal formulation of Compound I (ASR-Lipo) at 50 and 100 μM significantly reduced the increases in microvessel permeability induced by MIC-1 and PAF. Top graph is the overlap of the permeability changes from two individual experiments showing contrast effects of MIC-1 plus PAF on Lₚ in the present and absence of the nanoliposomal formulation in rat mesenteric vessels in vivo. The lower graph is the results summary from three groups of experiments in intact microvessels in vivo.

Like reference symbols in the various drawings indicate like elements.

### DETAILED DESCRIPTION

The following description of the disclosure is provided as an enable teaching of the disclosure in its best, currently known embodiments. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are part of the present disclosure. Thus, the following description is provided as illustrative of the principles of the present disclosure and not in limitation thereof.

### Definitions

Unless defined otherwise, all technical and scientific terms used herein have the same meaning as commonly understood to one of ordinary skill in the art to which this invention belongs. The following definitions are provided for the full understanding of terms used in the specification.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disuifonic, oxalic, isethionic, HOOC—(CH2)n- COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, the term “subject” or “host” can refer to living organisms such as mammals, including, but not limited to humans, livestock, dogs, cats, and other mammals.

Administration of the therapeutic agents can be carried out at dosages and for periods of time effective for treatment of a subject. In some embodiments, the subject is a human.

### Compound I

The present disclosure provides Compound I having the following formula:

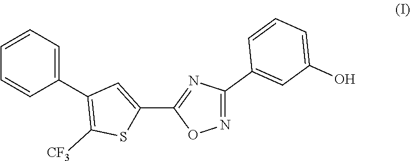 [SMILES: Oc1cccc(-c2noc(-c3cc(-c4ccccc4)c(C(F)(F)F)s3)n2)c1]

or a pharmaceutically acceptable salt, prodrug, or derivative thereof. Compound I is also referred as ASR396 in various parts of the present disclosure.

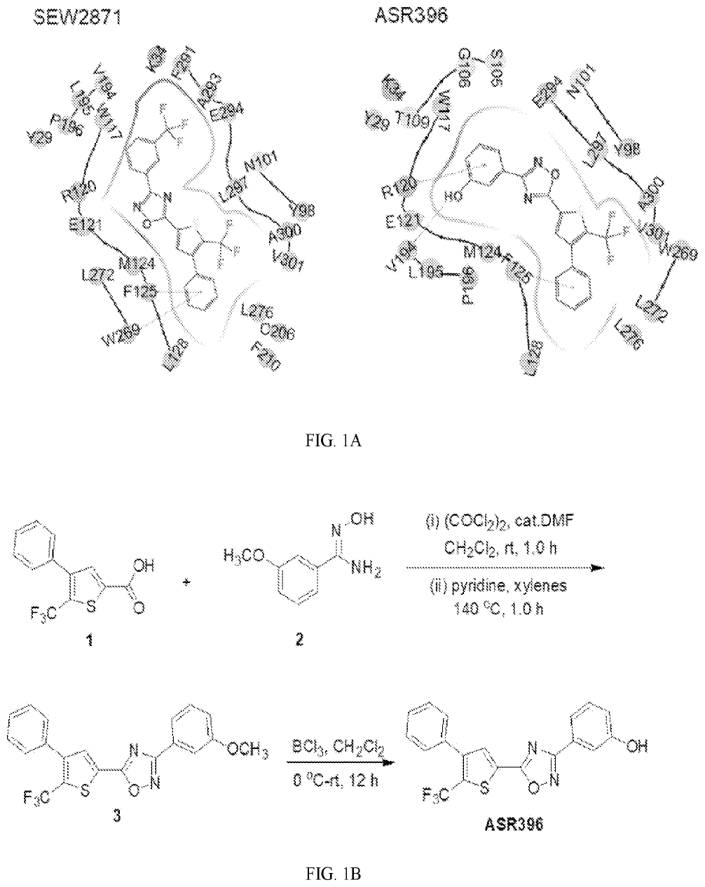

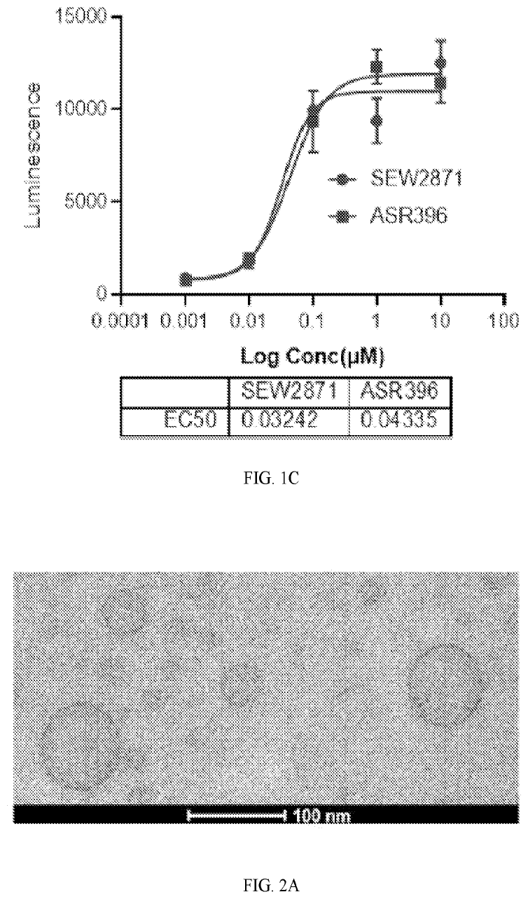

Compound I may be prepared according using a process according to the synthetic scheme provided in FIG. 1. A person of skill in the art would readily understand appropriate modifications that may be made to this process without undue experimentation, such as for example selection of reagents, solvent, reaction temperature, and reaction time, and therefore such modifications are considered part of the present disclosure.

### Pharmaceutical Compositions

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and an excipient of some sort may be useful in a variety of medical and non-medical applications. For example, pharmaceutical compositions comprising an active compound and an excipient may be useful for the treatment or prevention of an infection with a Mycobacterium.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

### Nanoliposomal Formulations

In some aspects, Compound I, or a pharmaceutically acceptable salt, prodrug, or derivative thereof, may be prepared as a nanoliposomal formulation.

Thus in one aspect, a formulation is provided comprises:

a plurality of nanoliposomes comprising a phospholipid and a PEG-phospholipid; and

Compound I, or a pharmaceutically acceptable salt, prodrug, or derivative thereof, encapsulated within the plurality of nanoliposomes.

In some embodiments, the phospholipid may be selected from a glycerophospholipid.

In some embodiments, the phospholipid may be selected from a phosphatidylcholine (PC), a phosphatidylethanolamine (PE), a phosphatidylserine (PS), a phosphatidylinositol (PI), a phosphatidic acid (PA), a phosphatidylglycerol (PG), and a cardiolipid (CL). In some embodiments, the glycerophospholipid may be a dimyristoyl, a dipalmitoyl, or a distearoyl glycerophospholipid. The phospholipid may be derived from an animal tissue source (such as bovine brain or egg yolk) or a vegetable oil source (such as soybean, corn, cotton seed, sunflower, or rapeseed oil). In some embodiments, the phospholipid is derived from egg yolk. In other embodiments, the phospholipid is derived from soybean oil.

In some embodiments, the phospholipid may be selected from 1,2-distearoyl-sn-glycero-3-phosphocholine (DSPC), 1,2-distearoyl-sn-glycero-3-phosphoethanolamine (DSPE), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1,2-dioleoyl-sn-glycero-3-phosphoethanolamine (DOPE), 1-palmitoyl-2-oleoyl-sn-glycero-3-phosphocholine (POPC), 1,2-distearoyl-sn-glycero-3-phosphoglycerol (DSPG), 1,2-dipalmitoyl-sn-glycero-3-phosphoglycerol (DPPG), 1,2-dioleoyl-sn-glycero-3-phospho-L-serine (DOPS), 1,2-dioleoyl-sn-glycero-3-phosphoglycerol (DOPG), 1,2-dipalmitoyl-sn-glycero-3-phosphoethanolamine (DPPE), 1-palmitoyl-2-oleoyl-sn-glycero-3-phosphoglycerol (POPG), 1,2-dieurocoyl-sn-glycero-3-phosphocholine (DEPC), 1-myristoryl-2-stearoyl-sn-glycero-3-phosphocholine (MSPC), 1,2-dierucoyl-sn-glycero-3-phosphoethanolamine (DEPE), 1-palmitoyl-2-oleoyl-sn-glycero-3-phosphoethanolamine (POPE), 1-palmitoyl-2-oleoyl-sn-3-phospho-L-serine (POPS), 1,2-distearoyl-sn-glycero-3-phospho-L-serine (DSPS), 1,2-dimyristoyl-sn-glycero-3-phosphoglycerol (DMPG), 1,2-dipalmitoyl-sn-glycero-3-phosphatidic acid (DPPA), 1,2-dimyristoyl-sn-glycero-3-phosphoethanolamine (DMPE), 1,2-dimyristoyl-sn-glycero-3-phospho-L-serine (DMPS), 1-stearoyl-2-lyso-sn-glycero-phosphocholine (S-LysoPC), 1,2-dipalmitoyl-sn-glycero-3-phospho-L-serine (DPPS), 1-stearoyl-2-oleoyl-sn-glycero-3-phosphocholine (SOPC), 1,2-dilinoleoyl-sn-glycero-3-phosphocholine (DLoPC), 1,2-didecanoyl-sn-glycero-3-phosphocholine (DDPC), 1-palmitoyl-2-lyso-sn-glycero-phosphocholine (P-LysoPC), 1-palmitoyl-2-stearoyl-sn-glycero-3-phosphocholine (PSPC), 1,2-dilauroyl-sn-glycero-3-phosphoethanolamine (DLPE), 1-myristoyl-2-palmitoyl-sn-glycero-3-phosphocholine (MPPC), 1-palmitoyl-2-myristoyl-sn-glycero-3-phosphocholine (PMPC), 1,2-distearoyl-sn-glycero-3-phosphatidic acid (DSPA), 1,2-dilinoleol-sn-glycero-3-phosphoethanolamine (DLoPE), 1-myristoyl-2-lyso-sn-glycero-3-phosphocholine (M-LysoPC), 1,2-dimyristoyl-sn-glycero-3-phosphatidic acid (DMPA), 1-oleoyl-2-lyso-sn-glycerophosphocholine (O-LysoPC), salts thereof (such as, for example, sodium salts or ammonium salts), and combinations thereof.

In some embodiments, the PEG-phospholipid is selected from 1,2-dimyristoyl-rac-glycero-3-methylpolyoxyethylene (DMG-PEG 2000 or DMG-PEG 5000), N-(methylpolyoxyethylene oxycarbonyl)-1,2-distearoyl-sn-glycero-3-phosphoethanolamine (DSPE-PEG 2000 or DSPE-PEG 5000), 1,2-distearoyl-rac-glycero-3-methylpolyoxyethylene (DSG-PEG 2000 or DSG-PEG 5000), N-(methylpolyoxyethylene oxycarbonyl)-1,2-dimyristoyl-sn-glycero-3-phosphoethanolamine (DMPE-PEG 2000), N-(methylpolyoxyethylene oxycarbonyl)1,2-dipalmitoyl-sn-glycero-3-phosphoethanolamine (DPPE-PEG 2000 or DPPE-PEG 5000), N-[2′,3′-bis(metylpolyoxyethylene oxy)propane-1′-oxycarbonyl]-1,2-distearoyl-sn-glycero-3-phosphoethanolamine (DSPE-2arm-PEG 2000 or DSPE-2arm-PEG 5000), 1,2-dipalmitoyl-rac-glycero-3-methylpolyoxyethylene (DPG-PEG 2000 or DPG-PEG 5000), 1,2-dioleoyl-rac-glycerol methoxypolyethylene glycol (DOG-PEG 2000 or DOG-PEG 5000), salts thereof (such as, for example, sodium salts or ammonium salts), or combinations thereof.

In some embodiments, the plurality of nanoliposomes have a molar % ratio of the phospholipid to the PEG-phospholipid greater than 50:50, for example a ratio of 55:45, 60:40, 65:35, 70:30, 75:25, 80:20, 85:15, or 90:10. In particular embodiments, the plurality of nanoliposomes have a molar % ratio of the phospholipid to the PEG-phospholipid of about 80:20.

In some embodiments, the present disclosure provides a formulation comprising: a plurality of nanoliposomes comprising L-α-phosphatidylcholine (ePC) and 1,2-dipalmitoyl-sn-glycero-3-phosphoethanolamine-N-[methoxy(polyethylene glycol)-2000]ammonium salt (DPPE-PEG 2000); and Compound I, or a pharmaceutically acceptable salt, prodrug, or derivative thereof, encapsulated within the plurality of nanoliposomes.

In some embodiments, the plurality of nanoliposomes may have an average diameter from about 50 nm to about 90 nm, from about 55 nm to about 85 nm, or from about 60 nm to about 85 nm. In some embodiments, the nanoliposomes may have an average diameter of about 50 nm, about 55 nm, about 60 nm, about 65 nm, about 70 nm, about 75 nm, about 80 nm, about 85 nm, or about 90 nm.

In some embodiments, the plurality of nanoliposomes may have a zeta potential of about 0.10 to about 0.40 mV, for example from about 0.20 to about 0.30 mV. In some embodiments, the plurality o nanoliposomes may have a zeta potential of about 0.10 mV, about 0.12 mV, about 0.14 mV, about 0.16 mV, about 0.18 mV, about 0.20 mV, about 0.22 mV, about 0.24 mV, about 0.26 mV, about 0.28 mV, about 0.30 mV, about 0.32 mV, about 0.34 mV, about 0.36 mV, about 0.38 mV, or about 0.40 mV

### Methods of Increasing Vascular Integrity/Decreasing Vascular Permeability

It is understood and herein contemplated that the compositions disclosed herein, for example, Compound I or a pharmaceutically acceptable salt, prodrug, or derivative thereof, either alone or as a pharmaceutical composition, in particular as a nanoliposomal formulation as described herein are effective in increasing vascular integrity/decreasing vascular permeability. Thus, in one aspect disclosed herein are methods of decreasing vascular permeability in a subject in need thereof, comprising administering a therapeutically effective amount of a compound of Compound I or a pharmaceutically acceptable salt, prodrug, or derivative thereof, either alone or as a pharmaceutical composition, in particular as a nanoliposomal formulation as described herein.

### Methods for Treating Diabetes

Increased vascular permeability is a complication that can arise in diabetes and lead to complications such as diabetic retinopathy. Thus, in one aspect disclosed herein are methods of treating, inhibiting, reducing, decreasing, ameliorating, and/or preventing diabetes, hyperglycemia, and/or complications arising from diabetes and/or hyperglycemia in a subject in need thereof, comprising administering a therapeutically effective amount of a compound of Compound I or a pharmaceutically acceptable salt, prodrug, or derivative thereof, either alone or as a pharmaceutical composition, in particular as a nanoliposomal formulation as described herein.

### Methods for Preventing or Inhibiting Metastasis

The present disclosure also provides for preventing metastasis of a cancer in a subject need thereof, the method comprising administering a therapeutically effective amount of Compound I, or a pharmaceutically acceptable salt, prodrug, or derivative thereof, either alone or as a pharmaceutical composition, in particular as a nanoliposomal formulation as described herein.

In another aspect, the present disclosure provides a method for inhibiting metastasis of a cancer in a subject in need thereof, the method comprising administering a therapeutically effective amount of the Compound I, or a pharmaceutically acceptable salt, prodrug, or derivative thereof, either alone or as a pharmaceutical composition, in particular as a nanoliposomal formulation as described herein.

A non-limiting list of different types of cancers for which metastasis can be inhibited or prevented by the disclosed methods is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, or cancers in general.

Representative examples cancers for which metastasis can be inhibited or prevented using the compound described herein include, but are not limited to: B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers, small cell lung cancer, non-small cell lung cancer, neuroblastoma, glioblastoma, ovarian cancer, pancreatic cancer, prostate cancer, skin cancers, melanoma, basal cell carcinoma, squamous cell carcinoma, liver cancer, squamous cell carcinomas of the mouth, throat, larynx, and lung, cervical cancer, cervical carcinoma, breast cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, AIDS-related lymphomas, or AIDS-related sarcomas.

Further examples of cancers for which metastasis can be inhibited or prevented by the disclosed methods include: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, and/or rectal cancers.

By way of non-limiting illustration, examples of certain embodiments of the present disclosure are given below.

### Examples

### Materials and Methods

### Source, Synthesis and Characterization of Compounds.

4-Phenyl-5-trifluoromethylthiophene-2-carboxylic acid (FIG. 1, compound 1) and N-hydroxy-3-methoxy-benzamidine (FIG. 1B, compound 2) were purchased from Millipore Sigma. The purity of Compound I (>99%) was verified by analytical high-performance liquid chromatography (HPLC) analysis by comparing the peak areas of the product relative to any impurities. Compound I was synthesized according to the procedures provided below:

### Synthesis and Chemical Characterization of Compound I

3-(3-Methoxy-phenyl)-5-(4-phenyl-5-trifluoromethyl-thiophen-2-yl)-[1,2,4]oxadiazole (FIG. 1B, intermediate 3) was synthesized as follows. To a solution of intermediate 1 (0.408 g, 1.5 mmol) in 5 mL of CH₂Cl₂ was added 1 mL of oxalyl chloride followed by 5 drops of DMF. The resulting mixture was stirred at room temperature for 1 hour and then concentrated. The resulting crude acid chloride was dissolved in 6:1 v/v xylenes/pyridine (7 mL) and to this N-hydroxy-3-methoxy-benzamidine (intermediate 2) (0.249 g, 1.5 mmol) was added. The resulting solution was heated at 140° C. for 1 hour and then cooled. The mixture was partitioned between 1:1 ethyl acetate/ether (50 mL) and 1N HCl (50 mL). The organic layer was separated, washed with 1N HCl (3×50 mL), saturated NaHCO₃ (50 mL) and brine (10 mL), dried over anhydrous MgSO4 and concentrated. The residue was purified by chromatography on silica gel using 8:2 v/v hexanes/ethyl acetate as the eluent to afford pure 3 as a white solid at a 75% (0.452 g) yield; m.p. 135-137 oC; ¹H NMR (500 MHz, CDCl₃): δ 3.93 (s, 3H), 7.11 (dd, J=8.0, 2.5 Hz, 1H), 7.44 (t, J=8.0 Hz, 1H), 7.46-7.51 (m, 5H), 7.69 (s, 1H), 7.77 (d, J=7.5 Hz, 1H), 7.93 (s, 1H); ¹³C NMR (125 MHz, CDCl₃) δ 170.0, 169.1, 159.9, 145.2, 134.4, 133.1, 130.7, 130.0, 128.9, 128.6, 127.4, 126.7, 125.1, 120.0, 118.6, 118.0, 112.1, 55.5; ESI-MS m/z 401 (M-H)⁺.

3-[5-(4-Phenyl-5-trifluoromethyl-thiophen-2-yl)-[1,2,4]oxadiazol-3-yl]-phenol (Compound I) was synthesized as follows. To a stirred solution of intermediate 3 (0.166 g, 0.412 mmol) in CH₂Cl₂ under nitrogen was added BCl₃ (6.18 mL, 6.18 mmol, 1M solution in CH₂C12) slowly at 0° C. The resulting solution was stirred for 12 hours at room temperature.

After completion of the reaction as indicated by TLC, the reaction was quenched with slow addition of H₂O (5 mL). The aqueous layer was extracted with CH₂C12 (2×30 mL), and the combined organic layers were washed with brine (10 mL), dried over anhydrous MgSO4 and concentrated. The residue was purified by chromatography on silica gel using 7:3 v/v hexanes/ethyl acetate as the eluent to afford the pure Compound I as a white solid at a 92% (0.147 g) yield; m.p. 131-133° C.; ¹H NMR (500 MHz, CDCl₃): δ 5.40 (br s, OH, 1H), 7.05 (ddd, J=8.0, 2.5, 1.0 Hz, 1H), 7.41 (t, J=8. Hz, 1H), 7.47-7.50 (m, 5H), 7.64-7.65 (m, 1H), 7.75 (dt, J=8.0, 1.0 Hz, 1H), 7.92 (d, J=1.5 Hz, 1H); ¹³C NMR (125 MHz, CDCl₃): δ 170.1, 168.8, 155.9, 145.3, 134.1, 133.1, 130.8, 130.5, 128.9, 128.6, 127.5, 126.6, 125.1, 120.2, 118.7, 118.6, 114.3; ESI-MS m/z 387 (M-H)⁺.

Compound melting points were recorded on a Fischer-Johns melting point apparatus and are uncorrected. ¹H NMR and ¹³C NMR spectra were recorded on a Bruker Advance 500 instrument in CDCl₃, operating at 500 and 125 MHz, respectively. Chemical shifts are reported in 6 values (ppm) and J values are reported in hertz (Hz). Experiments involving moisture and/or air sensitive components were performed under a nitrogen atmosphere in oven-dried glassware. Reagents, starting materials, and anhydrous solvents were purchased from commercial suppliers and were used as received. Reaction courses were monitored by thin-layer chromatography (TLC) on pre-coated silica gel 60 F254 aluminum sheets (Merck, Darmstadt, Germany), and the spots were visualized under UV light. The crude reaction products were purified by silica gel column chromatography using silica gel 60 Å (Merck, 230-400 mesh).

In Silico Binding Assessment of Compound I to Ensure Retention of Interaction with S1PR1.

Crystal structure of S1PR1 receptor (PDB ID: 3V2W) was prepared and the active site pocket assigned as described previously (26). The crystal ligand in the active site pocket was examined with the various designed S1PR1 agonists and the docking interaction scores were calculated using the Glide module of the Schrodinger software.

### Assessing Compound I Off-Target Binding.

The substructure-matching algorithm Erebus (erebus.doklab.org) was used to find proteins featuring a similar or identical binding site to S1PR1. Erebus is a substructure-matching platform that is based on a select query of inter-atomic distances, which define a structural motif on a bait protein, performs a search of the entire RCSB protein database for all proteins featuring a similar or identical scaffold (27). Using Erebus, off-target proteins from the RCSB database were identified by matching substructures with the same amino acids and atoms separated by the same distances (within a given tolerance) as the atoms of the query structure. The accuracy of a match was measured by the root-mean-square deviation (RMSD) or by the normal weight with a given variance.

### Development of a Nanoliposome Containing Compound I.

A 80:20 mol % L-α-phosphatidylcholine (ePC) and 1,2-dipalmitoyl-sn-glycero-3-phosphoethanolamine-N-[methoxy(polyethylene glycol)-2000] ammonium salt (DPPE-PEG-2000) at a final concentration 25 mg/mL along with Compound I (in methanol at 5 mg/mL) was utilized for manufacturing a nanoliposomal formulation of Compound I (Avanti Polar Lipids) (28). The solvent from the mixture was evaporated under N₂ gas and re-suspended in saline to make a final concentration of 5 mg/mL of the nanoliposomal formulation. The rehydrated mixture was heated at 60° C. for 60 minutes followed by sonication and extrusion through a 100 mm polycarbonate membrane.

### Estimation of Compound I Encapsulation in Nanoliposomal Formulation.

Drug encapsulation efficiency was performed using a filter separation process as described previously (28). Compound I not encapsulated in the nanoliposomes was separated by centrifugation for 30 minutes using a 10 kDa filter. Drug from the purified nanoliposomal formulation was extracted by nanoparticle destruction addition of a 1:1 chloroform and methanol solution. Precipitated lipids were removed by centrifugation and the Compound I concentration was extrapolated from a standard curve using a UV-visible spectrophotometer with a λₘₐₓ of 254 nm. Percentage of drug encapsulated was estimated as 100−(free drug after filter separation/total drug before separation)×100. Release kinetics were measured using dialysis through a 25 kDa membrane. Briefly, the nanoliposomal formulation was placed in a dialysis membrane and suspended in 1 L of a saline solution with constant stirring at 300 rpm for 120 hours. A 1-mL sample was taken from the dialysis bag at indicated time points, and the concentration of Compound I was measured by absorption at 254 nm after extracting with 1:1 chloroform:methanol.

### Stability of Nanoliposomal Formulation.

Stability of the nanoliposomes containing Compound I was performed as described previously (28). The nanoliposomal formulation was stored at 4° C. for indicated times. The size and charge of the nanoliposomes were calculated using a Malvern Zetasizer. The nanoliposomes were considered stable if there was less than a 5% change in the size and charge of the nanoliposomes.

### Confirmation of Nanoliposomal Formulation Structure and Size by Cryo-Electron Microscopy (Cryo-EM)

For vitrification, each TEM grid (QUANTIFOIL R 2/1, Electron Microscopy Sciences) with a thin layer of carbon film deposited over the holes was pre-treated by glow discharge. Samples (3.5 μl) were applied to the grid and vitrified using the vitrobot (Thermo Fisher) under the following condition: 4° C., 100% of humidity, and 3 seconds of blot time. Images were recorded by the electron microscope Arctica (Thermo Fisher) equipped with Ceta camera (4096×4096 pixels, Thermo Fisher). The imaging conditions was 92,000× in magnification, 4 in spot size, 2.7 mm in spherical aberration, at an accelerating voltage of 200 KV

### Assessment of 15-Day Repeated Dose Toxicity of Nanoliposomal Formulation.

Swiss-Webster mice were treated intravenously at indicated doses of the nanoliposomal formulation daily for 15-days and compared to control nanoliposomes lacking the agent. Changes in the animal body weights, motility and behavioral changes were monitored daily to measure body weight or behavioural related toxic effects (28). At the end of the 15-day treatment regimen, animals were euthanized and blood was collected to assess potential toxic effects. Serum samples were analyzed for levels of biomarkers that correspond to major organ functions as an assessment of toxic effects. Specifically, alanine aminotransferase, alkaline phosphatase, albumin, globulin, total protein, total bilirubin, blood urea nitrogen, glucose, creatinine, calcium and cholesterol were evaluated (29). Levels were compared to animals treated with nanoliposomes lacking Compound I, which served as the control.

### Cell Lines, Chemicals, and Culture Conditions.

The human melanoma cell lines, A375MGFP, UACC 903M-GFP and 1205Lu-GFP were maintained in DMEM (Invitrogen) supplemented with 5% FBS (Atlantic Biologicals) and GlutaMAX (Life Technologies). Human umbilical vein endothelial cells (HUVEC) were obtained from Lonza and maintained on gelatin-coated plates in endothelial growth media (EGM) supplemented with 2% FBS (Lonza). HEK293A-S1P1-GFP cells were maintained in DMEM with 10% FBS and 200 μg/ml G418 (30). Melanoma cells were used within 20 passages; HUVEC cells were used between passages 2-5. SEW2871 was purchased from Cayman Chemical and W146 was from TOCRIS. Bradykinin (BK) was obtained from Millipore Sigma and vascular endothelial growth factor (VEGF) and interleukin-8 (IL-8) were from Peprotech.

### HUVEC Cell Line Monolayer Model to Assess Gap Formation.

HUVECs were seeded on coverslips coated with fibronectin (Millipore Sigma) and cultured to confluency. The inhibitors of VEGF (nintedanib), ICAM-1 (A286982), PDE-4 (roflumilast), and mTOR (tacrolimus), and S1PR1 agonists, SEW2871 (DMSO final concentration 0.05%), Compound I (DMSO final concentration: 0.05%), and the nanoliposomal formulation of Compound I (saline) were added to HUVECs at 5 μM for 30 minutes before cells were stimulated with 0.5 μM BK for 30 minutes, 100 ng/mL VEGF or 150 ng/mL IL-8 for 1 hour. HUVECs fixed with 2% cold paraformaldehyde (Electron Microscopy Science) and treated with 0.1% triton X-100 (Fisher) were exposed to a human VE-cadherin antibody (Santa Cruz) followed by the secondary antibody conjugated with Alexa488 (Invitrogen). Coverslips were mounted to slides using the antifade mounting medium with DAPI (Vector Laboratories).

### S1PR1 Internalization Assay

HEK293A-21P1-GFP cells were plated on collagen-coated coverslips (30). Cells were cultured overnight in DMEM with 2% charcoal-stripped serum. Prior to the experiment, cells were cultured in serum-free DMEM for 1.5 hours. Cells were treated with 5 μM W146 for 30 minutes followed by 5 μM SEW2871 and Compound I. After washing with ice cold PBS, cells were fixed in 2% cold paraformaldehyde and mounted to slides using the antifade mounting medium with DAPI.

### Quantitative Measurements of Permeability Coefficient, Hydraulic Conductivity (Lp) in Individually Perfused Rat Mesenteric Microvessels.

Experiments were undertaken on mesenteric venules from female Sprague-Dawley rats (2-3-month old, 220-250 g body weight, Sage Laboratory). Inactin hydrate was used for anesthesia through subcutaneous administration. The initial dosage of inactin was 180 mg/kg body weight and an additional 5 mg/dose given as needed. After the rat reached the appropriate plane of anesthesia, determined by the loss of the toe pinch response and the righting reflex, a midline surgical incision was made in the abdominal wall. A loop of the ileum was gently extended from the abdominal cavity and the mesentery was spread over a pillar attached to an animal tray. The upper surface of the mesentery was continuously perfused with mammalian Ringer's solution at 37° C. All experiments were carried out on venules with diameters ranging between 40 to 45 m. Each experiment was performed on a single vessel with one experiment per animal. Euthanasia was performed through bilateral thoracotomy while animals were under anesthesia.

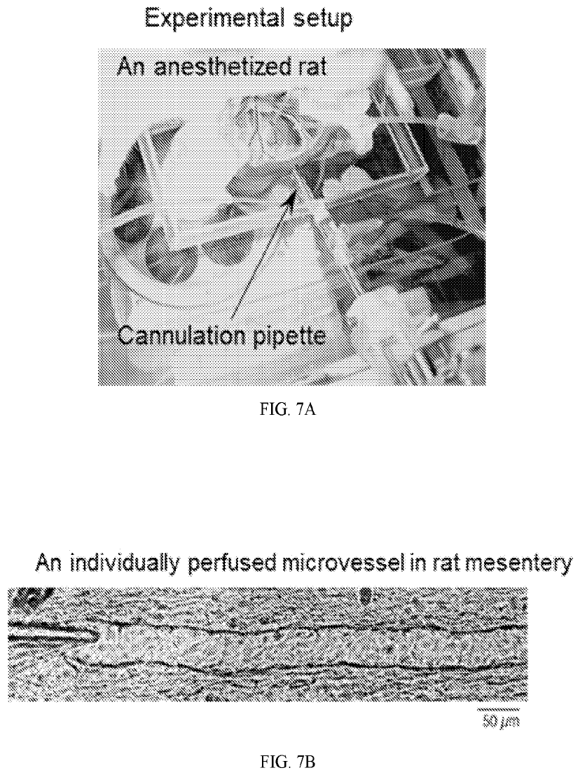

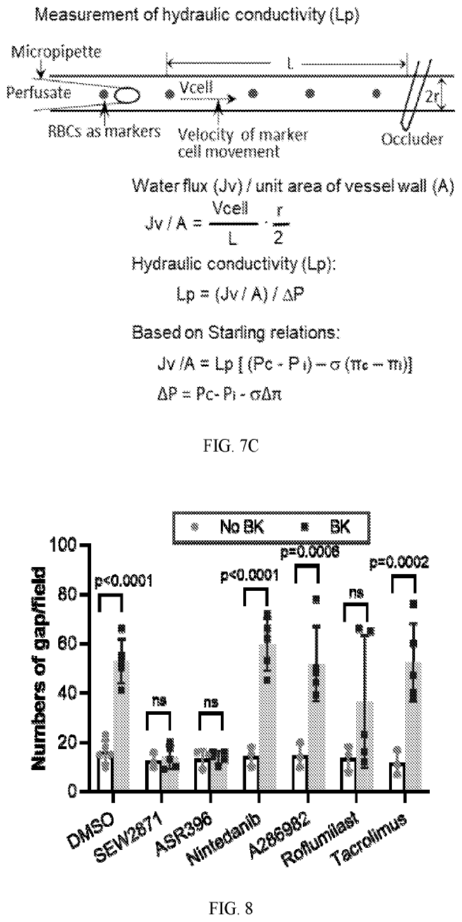

Microvessel permeability was assessed by measuring one of the permeability coefficients, hydraulic conductivity, Lp, using the modified Landis technique. The assumptions and limitations of the method have been evaluated in detail (31-33). Briefly, a single venular microvessel was cannulated with a glass micropipette and perfused with albumin-Ringer solution (control) containing 1% (v/v) rat red blood cells as markers. A hydrostatic pressure (range 40-70 cm H₂O), controlled by a water manometer, was applied through the micropipette to the microvessel lumen. The initial water flux (Jv) per unit area of microvessel wall (S), (Jv/S)₀, was calculated from the velocity of the marker cell after the vessel was occluded, the vessel radius, and the length between the marker cell and the occlusion site. Microvessel Lp was calculated from the Starling equation, Lp=(Jv/S)/ΔP, where ΔP represents the pressure difference between the hydrostatic pressure applied to the microvessel and the effective oncotic pressure generated from the albumin in the perfusate, assuming the tissue hydrostatic and oncotic pressures are negligible (31). In each experiment, baseline Lp and the stimulus-induced Lp changes were measured in the same vessel, which allows the changes to be compared with its own control. If Lp is relatively constant throughout the time course, the mean Lp value for each perfusate was calculated from all of the occlusions during that perfusion period. If a transient increase in Lp is observed, Lp is reported as the means of peak and sustained values. Details are shown in FIGS. 7A-7C.

### Assessment of Off-Target Effects Mediated by the Nanoliposomal Formulation on Key Signaling Pathways Using Western Blotting

A monolayer of HUVEC cells following varying treatments were lyzed in RIPA buffer containing protease and phosphatase inhibitors (Pierce Biotechnologies). 20 μg of cell lysate was used for western blotting and probed with antibodies according to the suppliers' recommendations to assess off target effects on several major signaling pathways. Antibodies to p-ERK, ERK, p-PI3K, PI3K, p-FAK, FAK, p-PAK1, PAK1, p-PAK2, PAK2, p-MLC, p-NF-κB, NF-κB were used to probe western blots (Cell Signaling). α-Tubulin (Millipore Sigma) was used as a loading control.

### Assessing Efficacy of the Nanoliposomal Formulation for Inhibiting Experimental Metastasis.

Athymic Foxn1^{nu} nude mice were intravenously injected with 15 mg/kg the nanoliposomal formulation of Compound I or nanoliposome lacking Compound I (Lipo) vehicle control, for 6, 12 or 18 hours prior to intravenous injection of 1 million A375M-GFP cells through the tail vein. For control comparison purposes, groups of nude mice were injected with 5 mg/kg SEW2871 or DMSO control intraperitoneally. For experiments using UACC 903M-GFP and 1205Lu-GFP cells, 15 mg/kg of the nanoliposomal formulation were given 6 hours before cancer cell injection. Body weight was monitored to ensure no significant toxicity during the experimental metastasis assay. On day 29, whole lungs were removed and imaged by fluorescent microscopy to quantify the numbers of metastatic nodules in a double-blinded manner.

### Statistical Analysis.

All values of Lp and tumor metastasis were means±SE. In vitro results were shown as mean±SD. Paired t-test (Nonparametric Wilcoxon matched-paired signed rank test, two-tailed) was used for paired experiments conducted in the same vessel, and Mann-Whitney test (two-tailed) was used for data comparison between two groups. One- and two-way ANOVA with Tukey post hoc test was used to compare data among multiple groups. A probability value of P <0.05 was considered statistically significant. Each “n” of microvessel perfusion experiment represents one experiment conducted in each rat.

### Results

### Synthesis of Compound I.

Compound I was synthesized as shown in FIG. 1B. 4-Phenyl-5-trifluoromethyl-thiophene-2-carboxylic acid (1) was first converted in situ to the corresponding acid chloride by treating with oxalyl chloride in the presence of catalytic N,N-dimethyl formamide. The resulting thiophene carboxylic acid chloride was further reacted with N-hydroxy-3-methoxy-benzamidine (2) in the presence of pyridine in xylenes at 140° C. for 1 hour to give the corresponding oxadiazole derivative 3 at a yield of 75% (34). The methoxy compound 3 on further treatment with BCl₃ in CH₂C12 at room temperature for 12 hours resulted in the corresponding hydroxy Compound I at a yield of 92% (FIG. 1B) (35). The structures of Intermediate 3 and Compound I were characterized on the basis of NMR and mass spectrometry. No significant change in the binding affinity of Compound I and SEW2871 to S1PR1 was demonstrated using an S1P1 GPCR cell-based agonist arrestin biosensor assay (Eurofins DiscoverX). SEW28731 and Compound I showed similar S1PR1 binding kinetics (FIG. 1C) with an EC₅₀ of 32 and 43 nM respectively, which suggested no change in binding due to the chemical modification.

### In Silico Predicted Off-Target Binding of Compound I Compared to SEW2871.

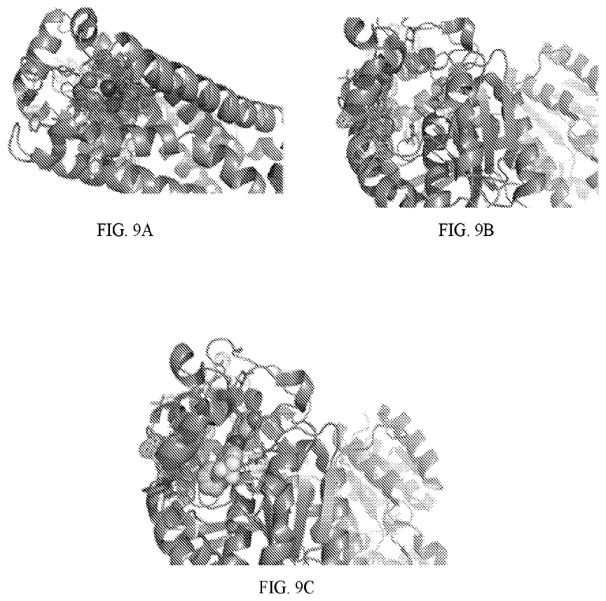

To predict whether the chemical modifications of SEW2871 led to any unexpected off-target interactions, the interaction plot of Compound I with S1PR1 was undertaken. This suggested that the binding scaffold of S1PR1 populated with R120, M124, F125, and V194 had significant interactions with Compound I. R1280 and F125 have been reported to be important for maintain the potency of SEW2871 (36). Hence, the side chain atoms of R120, M124, F125, and V194 engulfing Compound I in the binding pocket of S1PR1 were extracted and submitted as a query to Erebus. To precisely identify the most similar binding scaffolds to our query structure, a cut-off of RMSD≤5 Å to the query was imposed (Table 1). Erebus identified the query substructure itself from S1PR1 in the RCSB database (PDB IDs: 3V2Y, 3V2W) (FIG. 9A), which highlights the accuracy of the Erebus algorithm. Further, Erebus identified only one similar rigid binding scaffold, which was soluble epoxide hydrolase (PDB IDs: 3WKE, 3ANT) from Homo sapiens with RMSDs 3.83 and 4.98, respectively for two structures (FIG. 9B). To test whether the identified structural scaffold from soluble epoxide hydrolase had any binding affinity toward Compound I, Compound I was docked to the predicted site in epoxide hydrolase (PDB ID: 3WKE) using the MedusaDock suite, which is known for its rapid sampling efficiency and high prediction accuracy (37). The docked epoxide hydrolase-Compound I complex obtained from MedusaDock displayed strong interaction with Compound I in the Erebus predicted binding scaffold (FIG. 9C), which suggested off-target binding to Compound I. Given that the binding of Compound I to epoxide hydrolase is competitive with the native ligand, the physiological effect may not be pronounced due to strong binding of the endogenous epoxides to epoxide hydrolase (Table 2). Apart from these scaffolds, Erebus also identified similar structural scaffolds in proteins from microbial organisms Geobacillus thermoglucosidasius and Peptoclostridium difficile, which were omitted from the off-targets list since they were not mammalian.

TABLE 1
Off-Target Effects of Compound I

| PDB ID | Atoms | Residues | RMSD (Å) | Description | Organism |
|---|---|---|---|---|---|
| 3V27 | 32 | 10 | 0 | Crystal Structure of a | Homosapiens |
| | | | | Lipid G protein-Couple | |
| | | | | Receptor at 2.80 A | |
| 3V2W | 62 | 24 | 2.7 | Crystal Structure of a | Homosapiens |
| | | | | Lipid G protein-Couple | |
| | | | | Receptor at 3.35 A | |
| 3WKE | 42 | 19 | 3.83 | Crystal structure of | Homosapiens |
| | | | | soluble epoxide | |
| | | | | hydrolase in complex | |
| | | | | with t-AUCB | |
| 3V2W | 55 | 24 | 4.6 | Crystal Structure of a | Homosapiens |
| | | | | Lipd G protein-Coupled | |
| | | | | Receptor at 3.35 A | |
| 4C1P | 54 | 23 | 4.64 | Geobacillus | Geobacillus |
| | | | | thermoglucosidasius | thermoglucosidasius |
| | | | | GH family 52 | |
| | | | | xylosidase | |
| 3TDP | 52 | 20 | 4.89 | Crystal structure of | Peptoclostridium |
| | | | | Hydro Sulfide ion | difficile |
| | | | | Channel at pH 4.5 | |
| 3TDP | 50 | 20 | 4.94 | Crystal structure of | Peptoclostridium |
| | | | | Hydro Sulfide ion | difficile |
| | | | | Channel at pH 4.5 | |
| 3ANT | 41 | 18 | 4.98 | Human soluble epoxide | Homosapiens |
| | | | | hydrolase in complex with | |
| | | | | a synthetic inhibitor | |

TABLE 2
Binding Energy Comparison of Different Targets of Compound I

| System | Binding Energy (kcal/mol) | Notes |
|---|---|---|
| 2WKE with native ligand | −64.4 | |
| 2WKE with Compound I | −58.2 | |
| 3V2Y with native ligand | −45.3 | For Reference |
| 3V2Y | −56.6 | For Reference |

### Development and Physiochemical Characterization of the Nanoliposomal Formulation Containing Compound I.

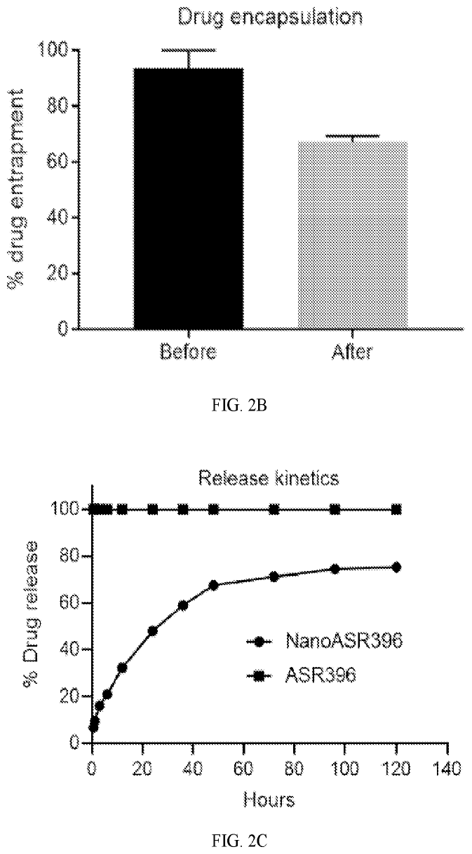

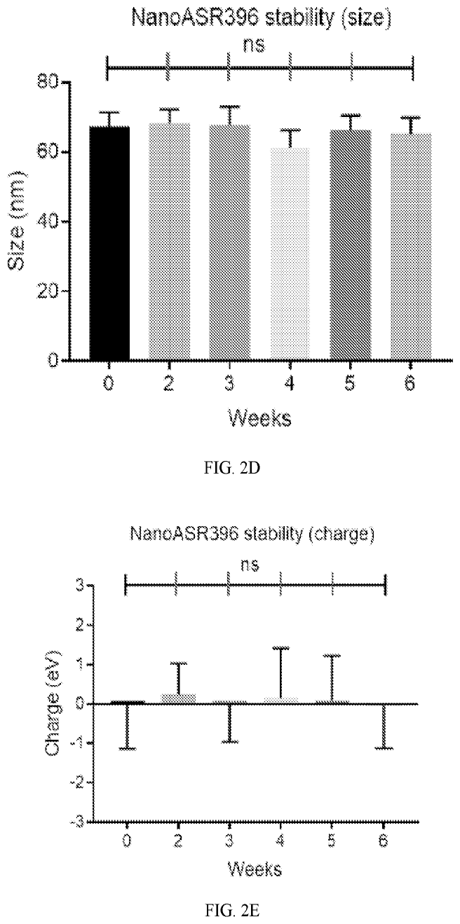

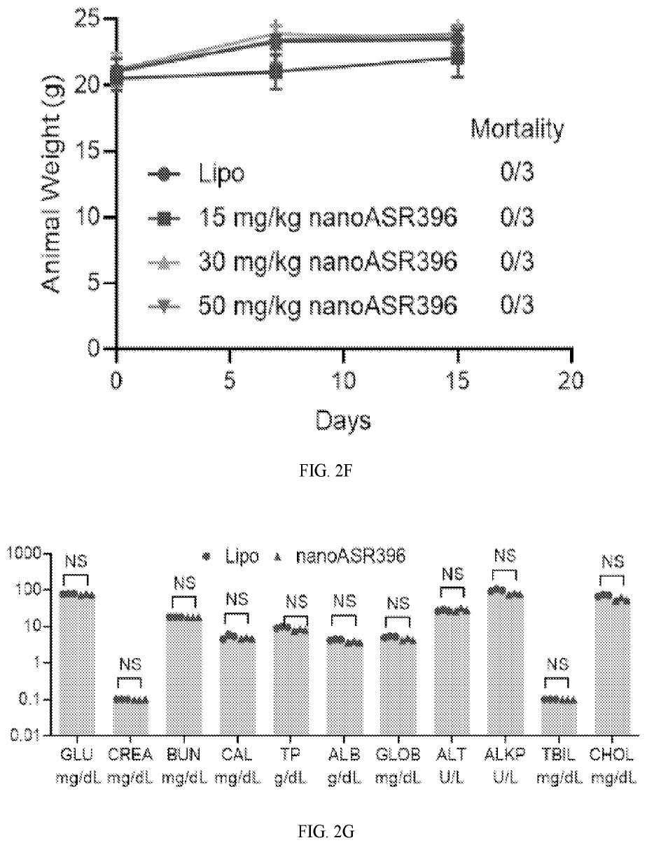

To identify a nanoliposomal formulation enabling optimal loading of Compound I, several formulations were examined and results for size, zeta potential charge, membrane potential and surface hydration were evaluated (data not shown). The optimal liposomal formulation selected contained the PEGylated nanoliposomal formulation made of 80:20 mol % ePC: DPPE PEG-2000. The average size of the nanoliposomes in saline ranged between 68.14±4.42 nm with a zeta potential of 0.24±0.02 mV. Cryo-electron microscopy demonstrated the spherical structure of the nanoliposomes and that the nanoparticles were not aggregations (FIG. 2A). Free Compound I was solubilized during nanoliposome preparation; however, a small amount of the drug was predicted to be loosely bound to the outer lipid layer of the nanoliposome. The amount of drug in the lipid shell was measured using the absorption maxima of the drug extrapolated from a standard curve generated from Compound I ranging from 0.01 and 0.1 mg/ml. The encapsulation efficiency for the nanoliposomal formulation after filtration was 67.1% compared to the nanoliposomal formulation without extrusion (FIG. 2B).

The drug release kinetics of Compound I was measured by dialyzing the nanoliposomal formulation in saline over 120 hours, upon which the dialyzed nanoliposomes were lysed and the remaining drug measured by extrapolation off a standard curve (FIG. 2C). Compound I was released from the nanoliposomes at a steady rate over 120 hours with approximately 70 to 75% release at 72 to 120 hours (FIG. 2C). The stability of refrigerated nanoliposomal formulation was examined weekly for a period of 6 weeks. Size and charge of the nanoliposomal formulation were estimated once every week. Both size (FIG. 2D) and charge (FIG. 2E) of the nanoliposomal formulation did not significantly vary over 6 weeks, indicating stability of the formulation. In order to evaluate the safety of the nanoliposomal formulation in animal models, a 14-day repeated range finder study was performed from 15-50 mg/kg intravenously administered daily. Treatment with the nanoliposomal formulation did not cause any significant change in animal weight (FIG. 2F) or changes in blood-based biomarkers of major organ related toxicity (FIG. 2G). Hence, the nanoliposomal formulation appeared to be a bioavailable agent with potential use in animals.

### The Nanoliposomal Formulation Attenuated Inflammatory Mediator-Induced Increases in Microvessel Permeability by Activation of Endothelial S1PR1.

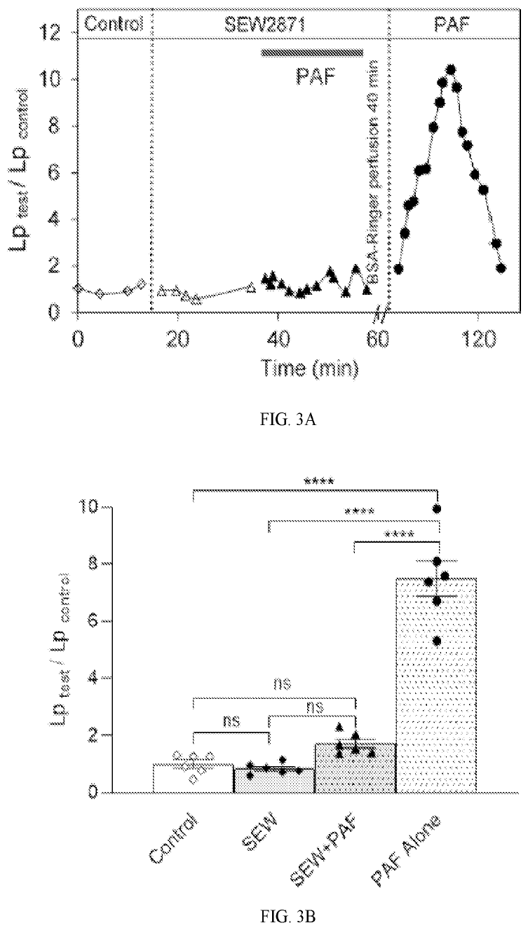

Previous studies conducted in individually perfused intact microvessels demonstrated that S1P had a potent inhibitory effect on inflammatory mediator-induced increases in microvessel permeability, mediated through the activation of S1PR1 (22,38). In this example, the effect and specificity of the S1PR1 agonist nanoliposomal formulation was examined on platelet activating factor (PAF)-induced permeability increases and results were compared to SEW2871 under identical experimental conditions. Permeability was assessed by measuring hydraulic conductivity, Lp, in individually perfused intact rat mesenteric venules. Each vessel was first cannulated and perfused with the control perfusate (1% bovine serum albumin, BSA, in Ringer's solution) to measure baseline Lp. The mean baseline Lp of 6 vessels was 1.14±0.16×10⁴ cm/s/cm H₂O. Perfusion of vessels with SEW2871 (10 PM) for 30 minutes did not change the baseline Lp. However, when each of the vessels was perfused with PAF (10 nM) in the presence of SEW2871, the PAF-induced Lp increase was significantly inhibited with the mean Lp value at 1.73±0.15 times that of the control. To test the vessel normal response to PAF, each vessel was given a second application of PAF after washing out SEW2871 with albumin-Ringer perfusion for 40 minutes. Then each vessel showed a typical PAF response with the mean peak Lp increased to 7.50±0.63 times that of the baseline. The time course of the Lp changes from an individual experiment was shown in FIG. 3A and the results summary was shown in FIG. 3B (n=6). These results indicated that pre-perfusion of vessels with SEW2871 prevented PAF-induced increases in microvessel Lp, functioning in a similar manner as S1P (22,38).

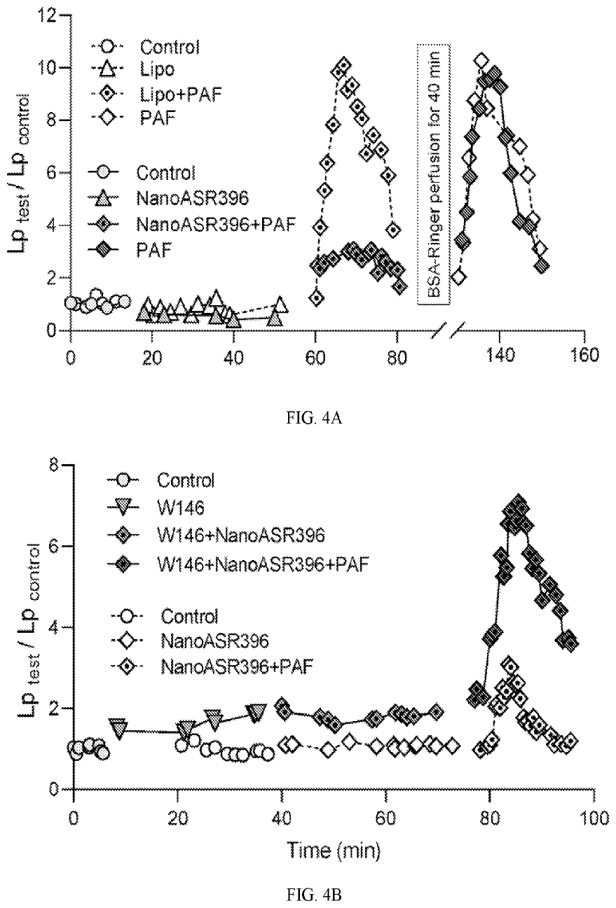

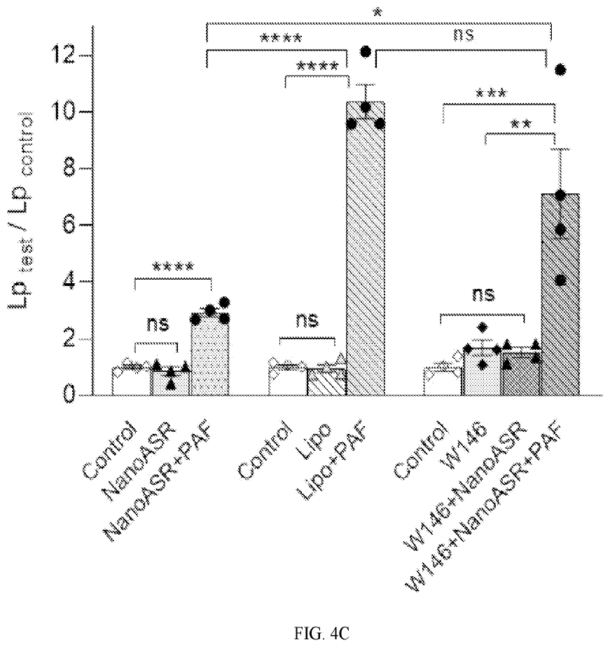

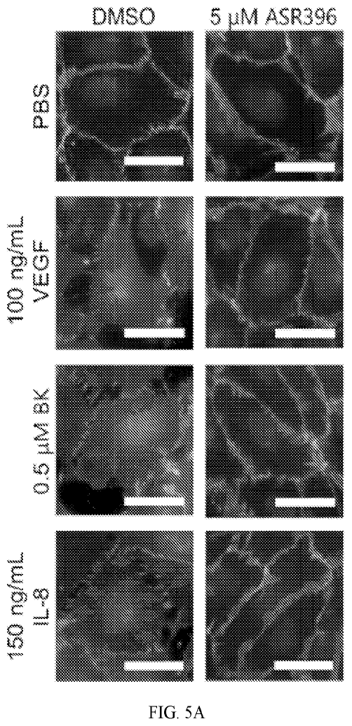

The effect of the nanoliposomal formulation on PAF-induced permeability increases were examined following the same experimental procedures as those used for SEW2871. The mean baseline Lp of 4 vessels was 1.72±0.13×10⁴′ cm/s/cm H₂O. Perfusion of the nanoliposomal formulation (100 μM) did not alter baseline Lp but significantly inhibited PAF-induced Lp increase with a mean peak Lp at 2.92±0.14 times that of the control value. In contrast, perfusion of empty nanoliposome alone showed no effect on PAF-induced Lp increase. Overlay of two-individual experiments with the application of the nanoliposomal formulation and nanoliposome alone demonstrated that the significant inhibition of PAM-induced Lp increase was due to the effect of Compound I and not empty nanoliposome (FIG. 4A).

The action of the nanoliposomal formulation as a specific S1PR1 agonist was examined in vessels that were pretreated with S1PR1 antagonist W146 before the application of the nanoliposomal formulation and PA. Inhibition of S1PR1 with W146 (10 μM) abolished the effect of the nanoliposomal formulation on PAM-induced Lp increases. In four W146-pretreated vessels, the PAM-induced mean peak Lp in the presence of the nanoliposomal formulation was 7.12±1.41 times that of the control; insignificant from the Lp responses to PAM in the absence of the nanoliposomal formulation. FIG. 4B shows the time courses of the Lp changes in the absence and presence of W146 from two individual experiments. The summary results of three groups of studies were presented in FIG. 4C (n=4 per group).

### The Nanoliposomal Formulation Inhibited Endothelial Gap Formation Mediated by Inflammatory Factors as Effectively as SEW2871 Acting Through the Myosin Light Chain Pathway.

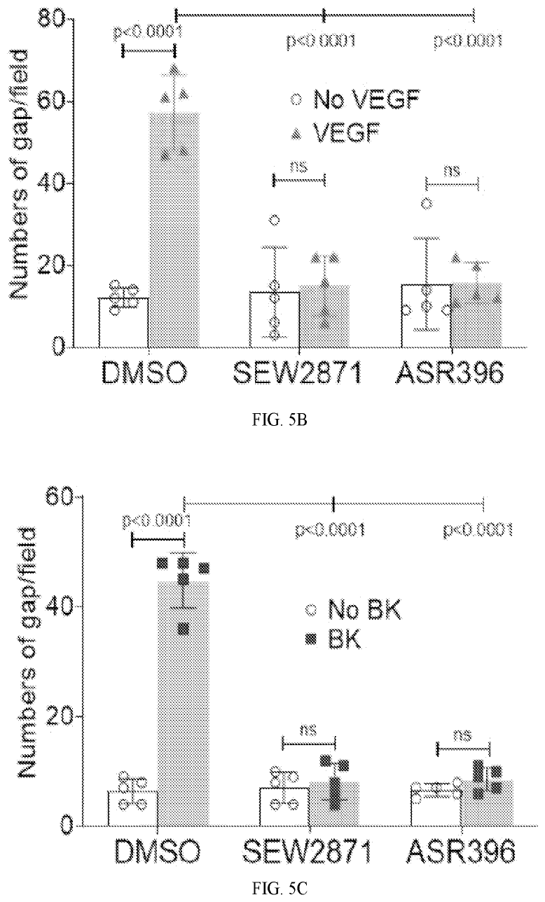

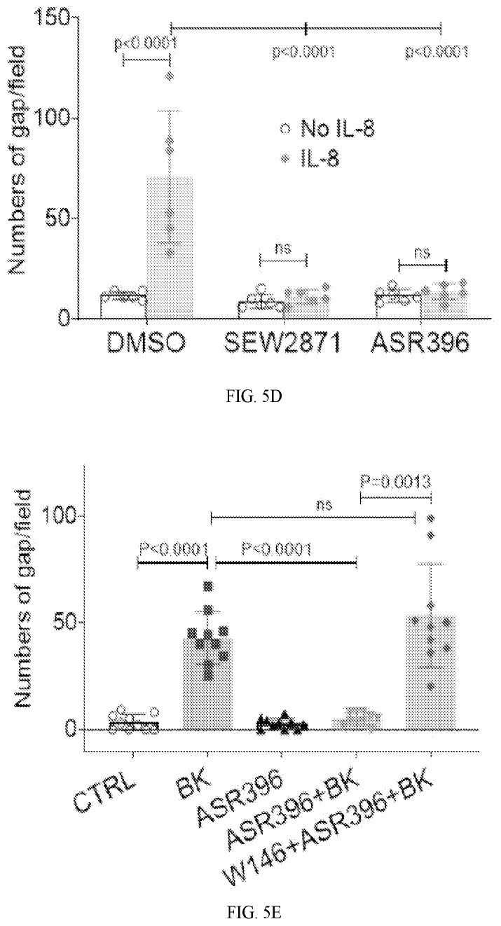

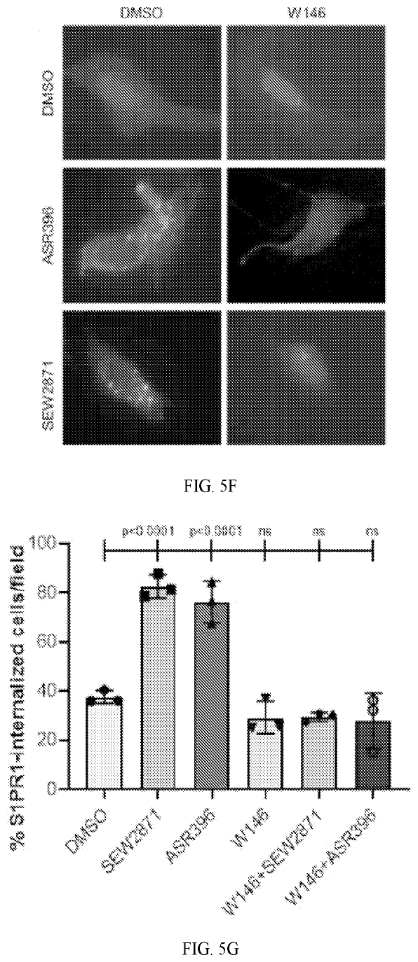

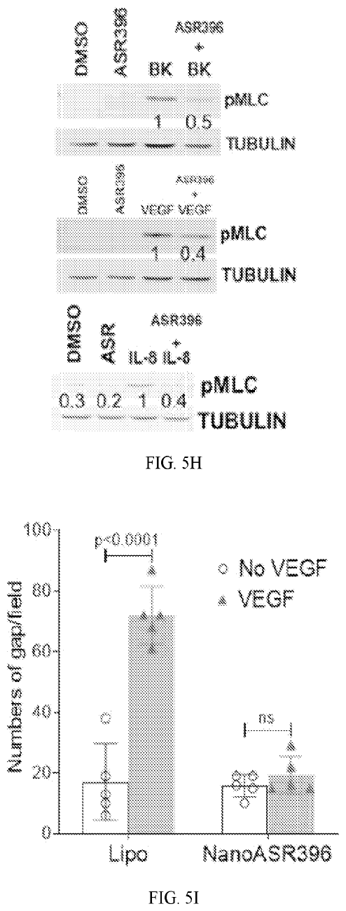

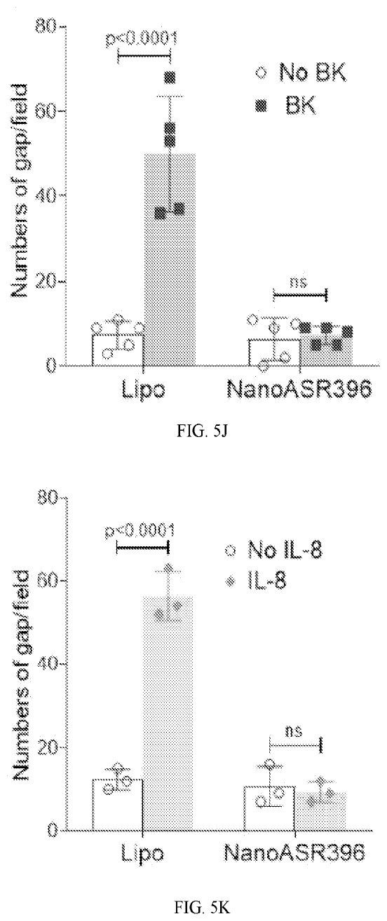

The effect of Compound I on endothelial permeability was further confirmed using a cultured EC monolayer model. As reported by other groups and shown in FIG. 5A, vehicle treated ECs in this model had a continuous VE-cadherin junctional protein line between ECs. When treated with VEGF, ECs dissociated from each other to form gaps with a discontinuous distribution line of VE-cadherin (FIG. 5A). Compound I treatment alone showed a similar result to that observed with the untreated control (FIG. 5A). ECs when treated with both Compound I and VEGF showed no gap formation with an intact VE-cadherin line at junctions between ECs (FIG. 5A). Quantitation of the gap formation mediated by VEGF (FIG. 5B), demonstrated that Compound I had similar gap inhibitory efficacy to SEW2871.

Compound I efficacy for inhibiting gap formation by inflammatory mediators BK and IL-8 was also examined (FIGS. 5A, 5C, and 5D). As predicted, ECs treated with BK (FIG. 5A), and IL-8 (FIG. 5A) had increased numbers of gaps, while treatment with either Compound I or SEW2871 similarly inhibited this effect (FIGS. 5C, 5D and 5E). Thus, Compound I and SEW2871 similarly blocked gap formation suggesting that Compound I retained the functional activity of the parent compound.

To show that Compound I was activating the S1PR1 to prevent gap formation, HUVECs were treated with W146 (S1PR1 antagonist) before treatment with Compound I and BK. W146 pretreatment abolished the inhibitory effect of Compound I on BK-induced gap formation (FIG. 5E), suggesting that Compound I-mediated activation of S1PR1 was responsible for inhibiting gap formation in the EC layer.

A S1PR1 internalization assay using HEK293A-S1P1-GFP cells was undertaken to show that Compound I and SEW2871 similarly enhanced S1PR1 internalization, leading to its accumulation in the cytoplasm. Approximately 37% of cells in the DMSO treatment has internalized S1PR1 while more than 75% of cells in the Compound I (FIG. 5F) and SEW2871 (FIG. 5G) treatment had internalized S1PR1 in the cytoplasm. Internalization mediated by Compound I and SEW2871 could be blocked using the S1PR1 antagonist W146, suggesting efficacy of both agonists for activating S1PR1 (FIGS. 5F and 5G).

MLC phosphorylation is critical in maintaining endothelial barrier integrity (18). HUVECs were treated with BK, VEGF or IL-8 and pMLC levels were compared by western blotting to treatment with Compound I. Compound I treatment alone did not increase pMLC levels but treatments of BK, VEGF or IL-8 significantly increased pMLC levels (FIG. 5H). Compound I was able to reverse and decrease the pMLC levels suggesting that the gap formation was involved in the MLC signaling and reversal effect of Compound I.

Compound I was able to inhibit gap formation mediated by inflammatory mediators VEGF (FIG. 5I), BK (FIG. 5J), and IL-8 (FIG. 5K) compared to EL, similar to that observed with Compound I in DMSO. No significant difference was observed in EC gap formation between HUVECs treated with Compound I or its nanoliposomal formulation prior to BK, VEGF and IL-8 exposure. At the concentration of 5 μM, SEW2871, Compound I and its nanoliposomal formulation showed similar potencies to inhibit gap formation. Thus, the nanoliposomal formulation did not affect the activity of the compound.

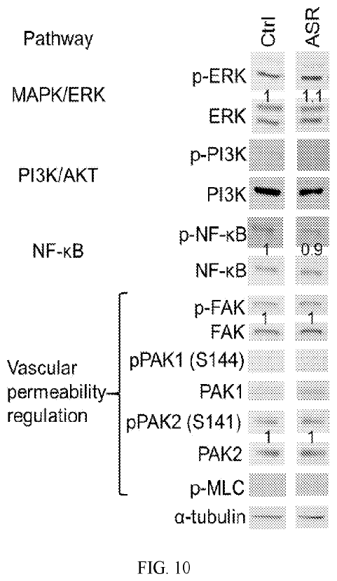

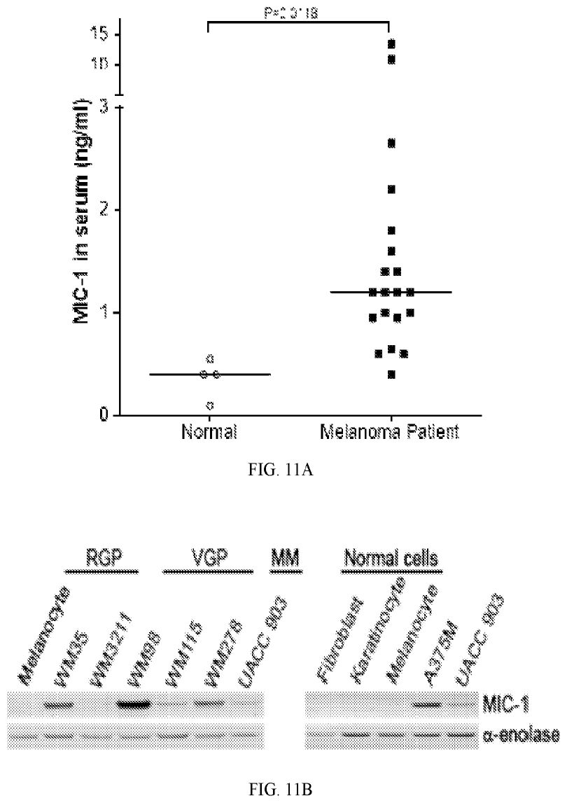

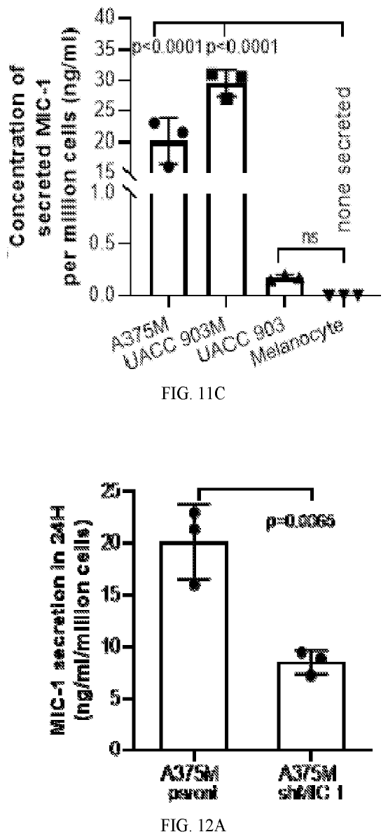

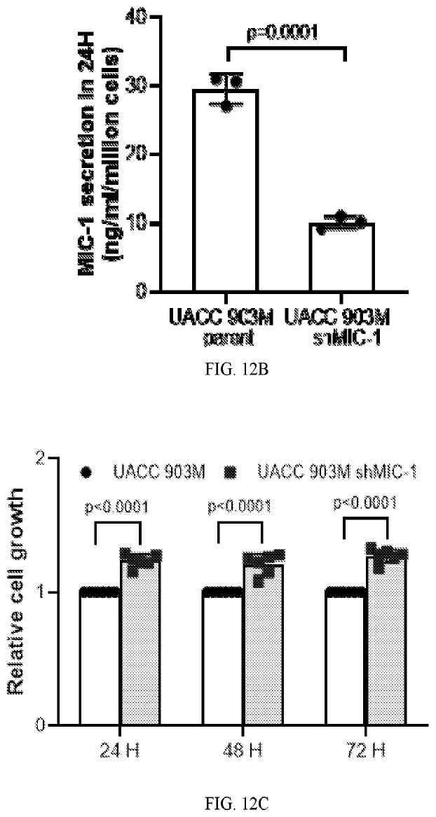

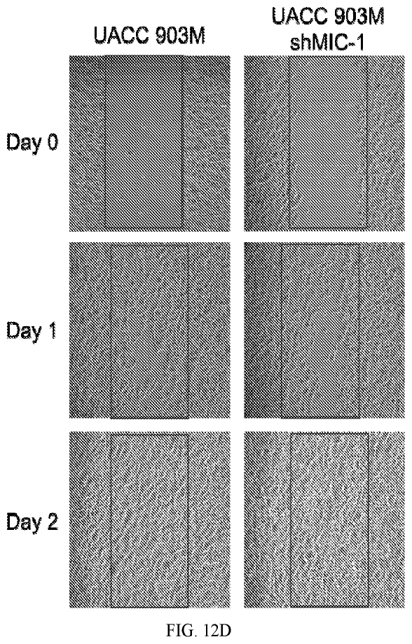

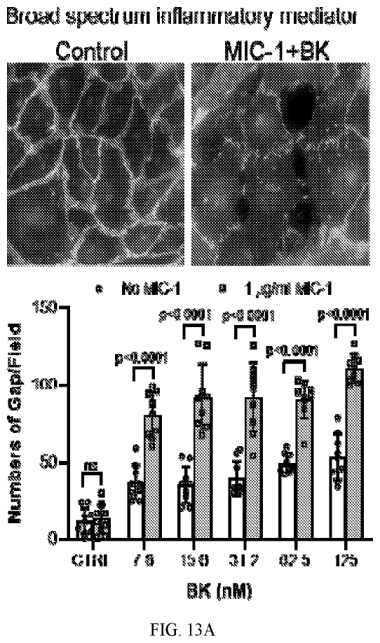

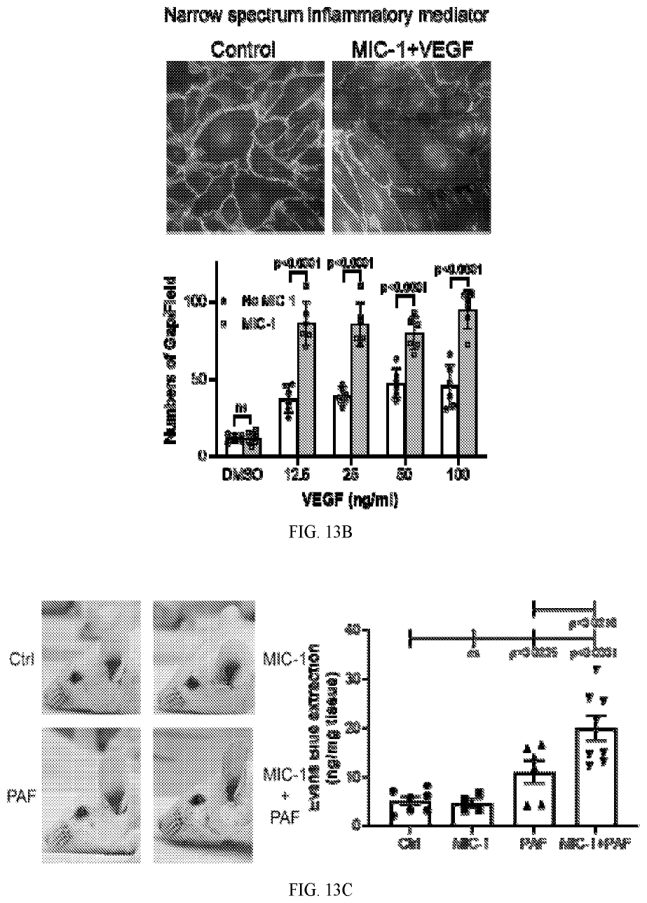

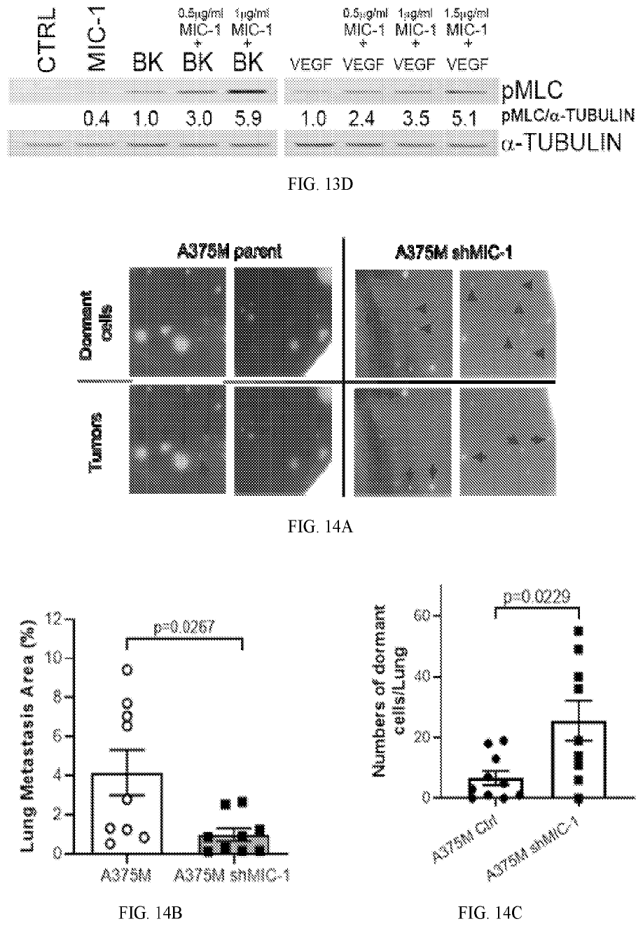

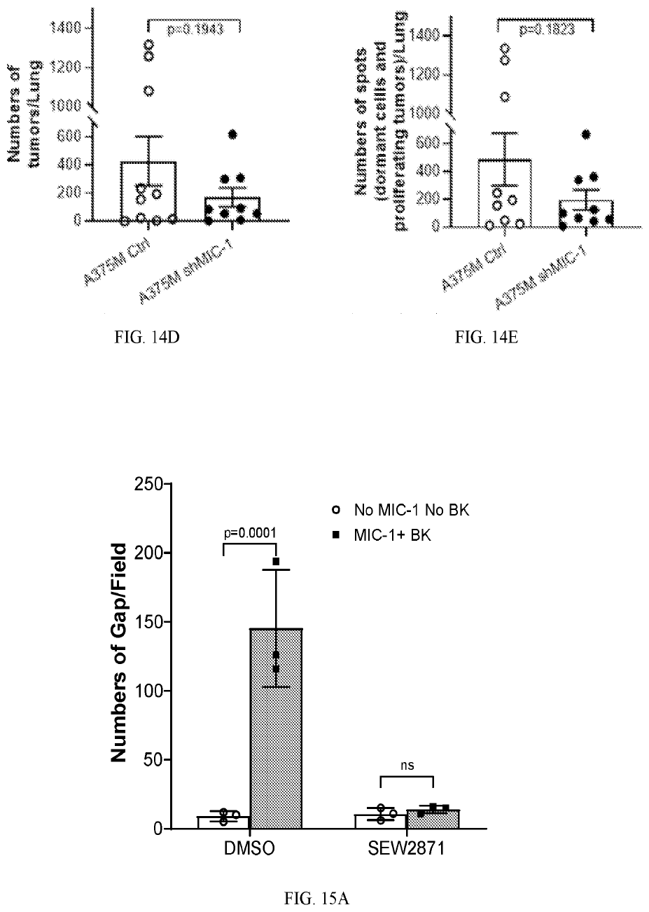

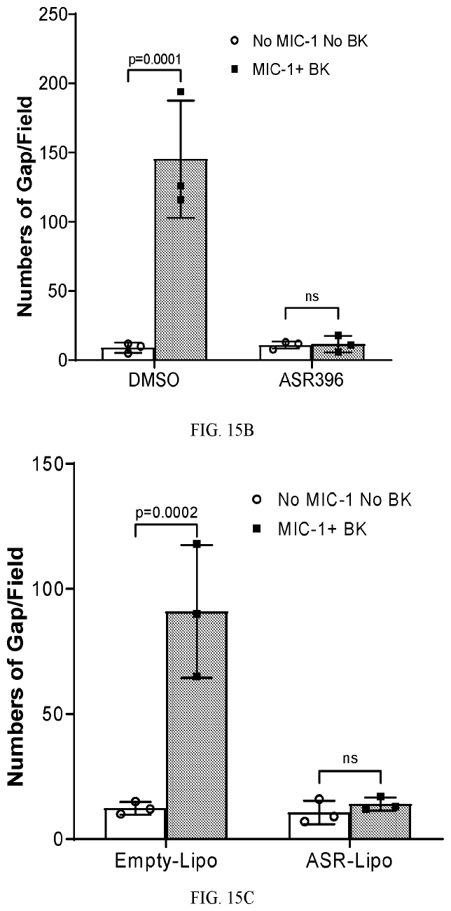

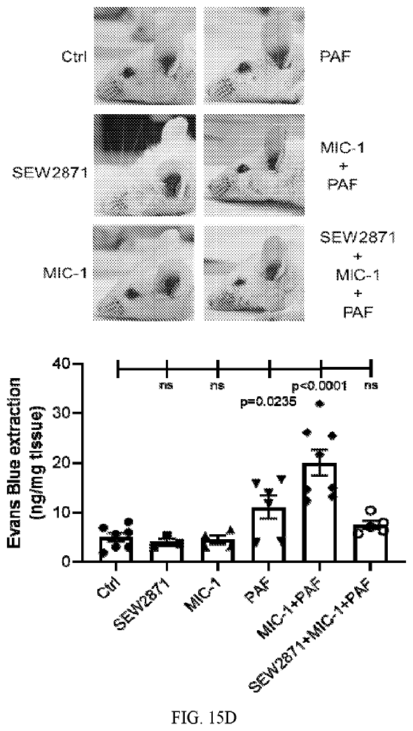

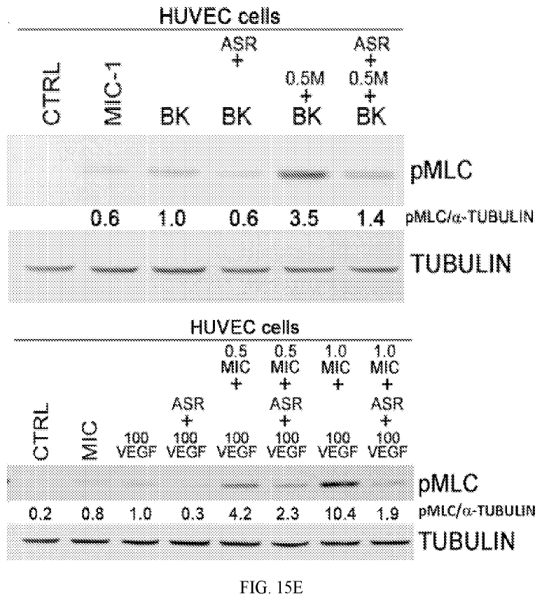

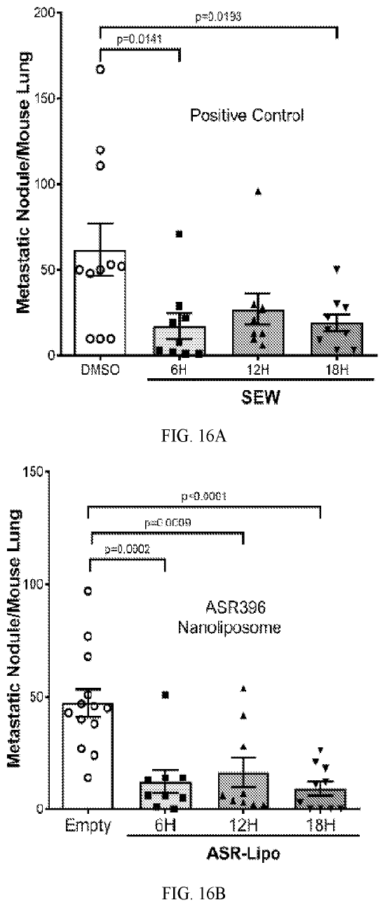

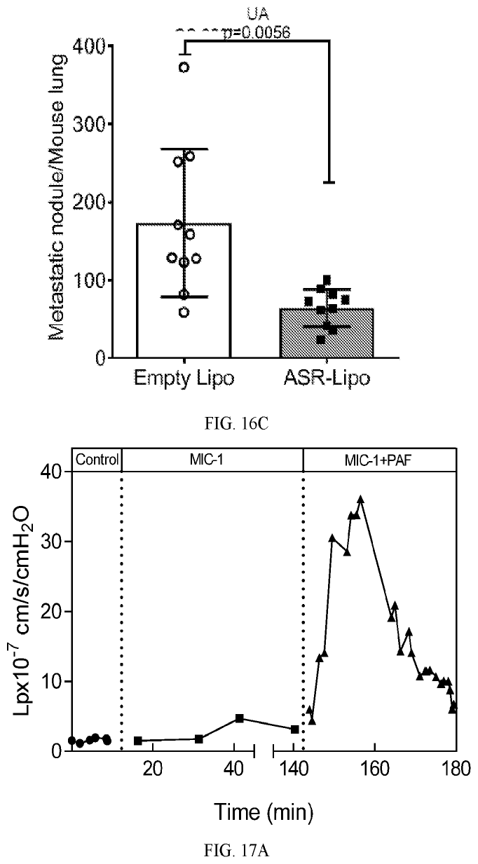

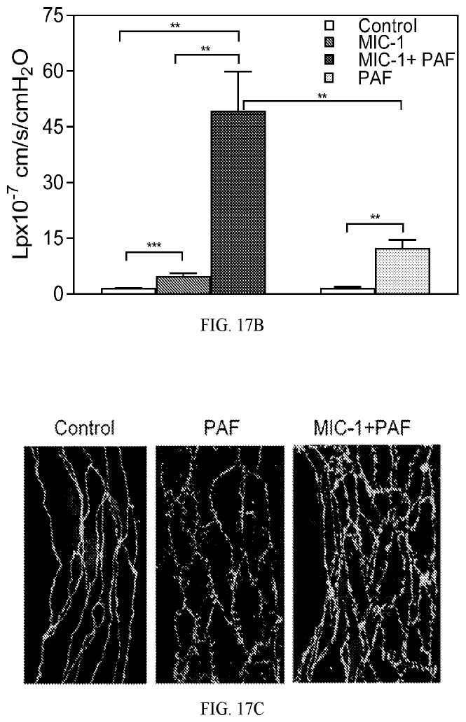

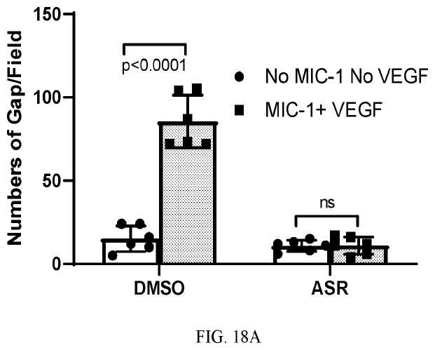

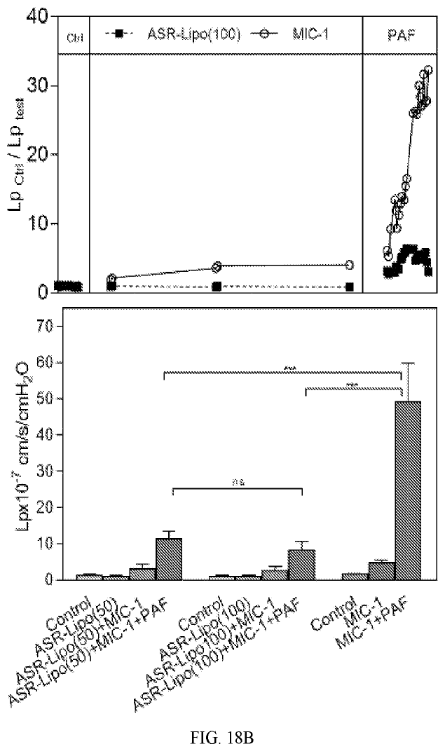

To determine whether Compound I induced off-target effects in HUVEC cells, the effects of the compound were examined on several signaling pathways important in melanoma development, including the MAPK/ERK, PI3K/AKT, NF-1κB, and the vascular permeability regulation pathways. No significant changes in levels or activity of biomarker proteins in these pathways were detected between the control and Compound I treated ECS (FIG. 10) suggesting that Compound I did not trigger significant off-target effects.

The Nanoliposomal Formulation Inhibits Metastasis Similar to that Occurring with Control SEW2871.

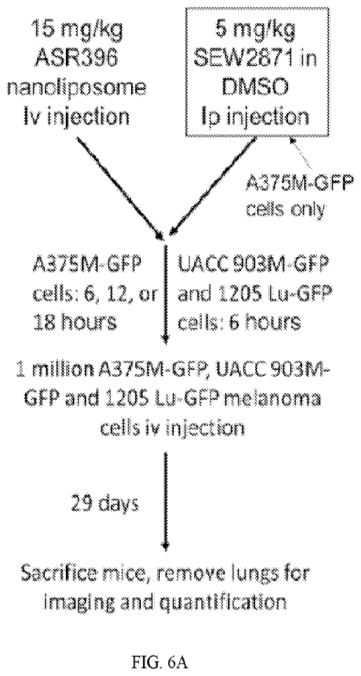

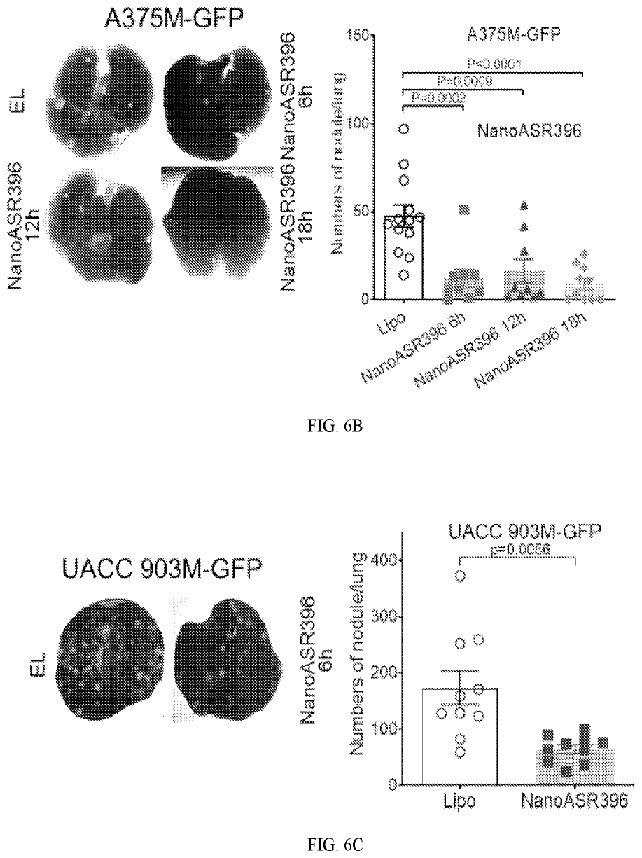

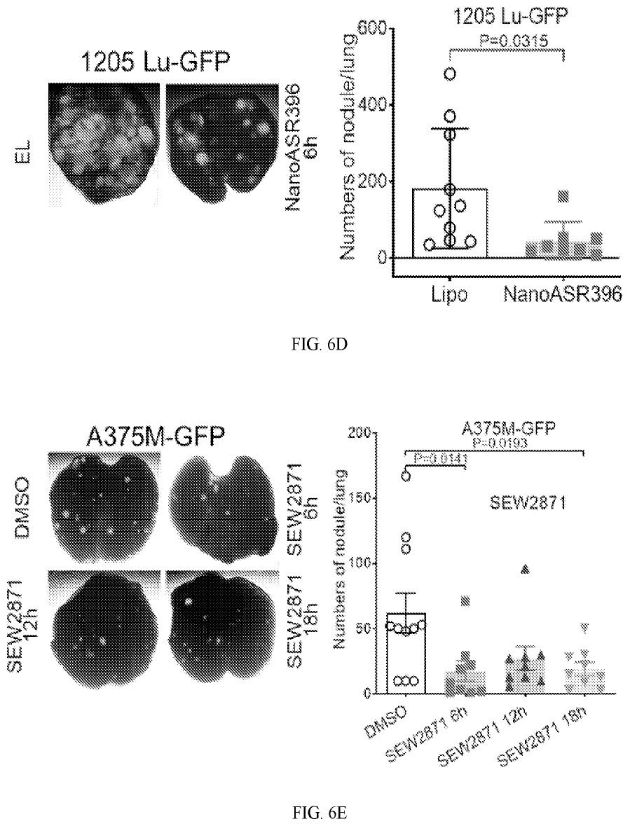

To test the efficacy of the nanoliposomal formulation for inhibiting melanoma metastasis, an experimental metastasis mouse model was used and the treatment protocol summarized in FIG. 6A. Mice were treated with the nanoliposomal formulation intravenously prior to injecting GFP-tagged A375 M, UACC 903 M or 1205 Lu melanoma cells. Control mice for the A375 M cell line developed 45 visible A375M-GFP melanoma metastatic nodules in the lung whole mice treated with the nanoliposomal formulation developed only 9-16 metastatic nodules, which was a 65-80% reduction (FIG. 6B). Nanoliposomal formulation treatment also led to a 62.7% reduction in UACC 903M-GFP (FIG. 3C) and 79.1% reduction in 1205 Lu-GFP (FIG. 6D) melanoma cell-mediated metastasis confirming this efficacy. To compare the effects of the nanoliposomal formulation to SEW2871 on metastasis inhibition, mice were treated with SEW2871 intraperitoneally before the administration of A375M-GFP melanoma cells. Control mice developed 62 visible A375M-GFP melanoma metastatic nodules in the lungs, while mice treated with SEW2871 developed 17-27 metastatic nodules (FIG. 6E). Thus, mice treated with the nanoliposomal formulation had melanoma metastasis in the lung reduced up to 80%, which was similar to that observed with SEW2871, suggesting it could be a potentially effective metastasis inhibitor.

### DISCUSSION

S1P can potently inhibit inflammatory mediator-induced increases in microvessel permeability through the activation of S1PR1 (22,38). Based on those observations, we developed a new nanoparticle S1PR1 agonist, the nanoliposomal formulation of Compound I, to specifically reverse the cancer-associated vascular inflammation that promotes metastasis. Studies conducted in both intact microvessels and a cultured EC monolayer model demonstrated its potent efficacy for enhancing endothelial barrier function, inhibition of permeability increases, and EC gap formation mediated by a variety of inflammatory mediators as well as tumor cell secreted cytokines. Significantly, the effect of the nanoliposomal formulation inhibited the inflammatory mediator-induced permeability increases using a quantitative measure of the permeability coefficient, L_{P}, in intact rat microvessels. The nanoliposomal formulation inhibited PAF-induced permeability increases similar to that observed with the SEW 2871 control. Furthermore, the action of the nanoliposomal formulation was abolished by pretreating vessels with a S1PR1 antagonist, W146, which confirmed the effect of the nanoliposomal formulation occur through S1PR1 activation in ECs. Most importantly, systemic application of the nanoliposomal formulation of Compound I inhibited lung metastasis by up to 80% in a melanoma metastasis animal model. Results demonstrated that the action of the nanoliposomal formulation not only recapitulated the effect of S1P on EC barrier integrity, but also minimized the potential confounding effects of S1P on the activation of other S1P receptors, suggesting its clinical potential for retarding the metastatic spread of cancer.

VEGF, BK and IL-8 are some of the inflammatory cytokines that are elevated in the serum of advanced-stage melanoma patients and in animal models containing melanomas (13). These cytokines can significantly alter the architecture of VE-cadherin and the phosphorylation status of MLC in endothelial cells leading to the detachment of these cells from one another to form gaps (39, 40). VEGF and BK are well-known permeability inducers and mechanisms mediating this process has been reviewed in detail (41). The IL-8 and CXCR1/2 complex has been shown to increase vascular permeability by transactivating VEGFR2 (42). It is almost impossible to target each individual inflammatory cytokine in order to prevent their effects on EC integrity. Therefore, our approach of directly enhancing the EC barrier integrity to prevent gap formation-mediated by a wide-spectrum of inflammatory cytokines could effectively reduce cancer cell metastasis for many cancer types and not just melanoma.

Compound I was developed to be loaded into a nanoliposome for bioavailable intravenous delivery. The chemical structure of Compound I was found to have better binding interaction in the S1PR1 active pocket with a higher docking score compared to SEW2871. Compound I was also confirmed to specifically interact in the active pocket of S1PR1 using the Erebus algorithm. Binding of Compound I to S1PR1 could be blocked using the S1PR1 antagonist W146, which functionally demonstrated that the new compound maintained its binding to the receptor. Thus, the data suggested that Compound I was a specific S1PR1 agonist.

A major advantage of Compound I is that it can be encapsulated into nanoliposomes while SEW2871 cannot. In recent years, nanotechnology has been used to more effectively deliver agents to treat cancer by reducing toxicity, increasing bioavailability and prolonging the time in the circulation (43). Nanoparticles can increase drug solubility, improve pharmacokinetics and minimize side-effects (43). Nanoliposomes, compared to the traditional drug delivery route, have some promising benefits, including better drug delivery, protection of active drugs from environmental factors, prevention of early degradation of the drug, cost-effective formulation for expensive agents and more efficient treatments with less toxicity (44). Moreover, the biodegradability and biocompatibility of nanoliposomes make them excellent therapeutic vehicles (44). Using a melanoma mouse model, we have previously demonstrated the low toxicity, high bioavailability and prolonged release of the nanoliposomal forms of anti-melanoma drugs, such as leelamine (45), arachidonyl trifluoromethyl ketone (46), aldehyde dehydrogenase inhibitors (47), celocoxib (48), and plumbagin (48).

The nanoliposomal formulation increased bioavailability by enabling direct injection into the vascular system wherein it would have immediate access to the ECs where it needed to act. The nanoliposomal formulation inhibited EC gap formation in the vascular system, which led to fewer melanoma lung metastases. We have previously shown that melanoma cells secrete IL-8 to attract neutrophils which tether them to the vascular endothelial cell layer to promote metastasis (49). The results of this study extend this process by showing that the cytokines secreted by these cells can further aid in this process by enabling gap formation in the EC layer through which the cancer cells can extravasate to promote metastasis. This process can be inhibiting using the bioavailable nanoliposomal formulation, which releases the S1PR1 agonist at the EC layer to enhance vascular barrier integrity. Thus, it is possible to counteract the effects of inflammatory cytokines non-specifically and provide better protection against these factors to maintain EC homeostasis to inhibit inflammation-mediated melanoma metastasis.

In normal endothelial cells, the S1P/S1PR1 complex enhances junctional assembly (50) while at high concentration of S1P, the S1P/S1PR3 complex in endothelial cells disrupts the barrier structure (51). An S1PR1/3 agonist, FTY720, has been developed but has the side-effects of inducing bradycardia (52), macular oedema, cardiovascular complications, and brain inflammation (53). This occurs following the activation of S1PR3 (52) or S1PR1 and S1PR3 (53). Since Compound I is a specific agonist to S1PR1, these toxic effects would not likely be observed.

S1P through the S1P receptors has been reported to trigger a possible epithelial-to-mesenchymal transition (EMT), leading to increase cell migration, invasion and metastasis in breast, liver and melanoma (54-56). In melanoma, the main S1P receptor regulating the S1P-mediated EMT and cell invasion has been reported to be S1PR2 (54), therefore, using the nanoliposomal formulation would likely avoid this concern. The S1PR1 pathway has been reported to be involved in S1P-mediated lymphangiogenesis, which indicated a potential role in lymphatic cancer metastasis (57). A recent study showed that S1PR1 plays a crucial role in high-molecular-weight-hyaluronan-mediated permeability decrease in cancer lymphatic endothelial cell monolayer (58). Effects, if any, of Compound I on melanoma lymphatic metastasis remains to be examined. However, since surgical cancer excision has potential to disseminate cancer around the patient's body (59), a single treatment of the naoliposomal formulation might be sufficient for use prior to melanoma surgery to enhance endothelial cell junctional integrity, in order to reduce surgery-mediated cancer spread. Since this treatment modality would only require a single administration of the nanoliposomal formulation, this approach could reduce the concerns related to potential EMT-mediated effects occurring with long term treatment.

In summary, this report suggests that multiple inflammatory cytokines secreted from melanoma cells can create gaps in the EC layer, which can subsequently promote metastasis. To combat vascular endothelial inflammation mediated by these factors, a novel S1PR1 agonist Compound I has been developed, which could reverse the collective effect of these proteins. Compared to SEW2871, Compound I exhibited several significant advantages including better association with S1PR1 and loadability into a nanoliposome. The nanoliposomal formulation of Compound I had an average size of 69 nm, neutral-charge in saline and was stable for 6 weeks when refrigerated. The nanoliposomal formulation significantly prevented permeability increases and gap formation in the vascular endothelium and an EC monolayer model induced by inflammatory cytokines. The nanoliposomal formulation enhanced endothelial cell layer integrity by decreasing the phosphorylation of MLC. As a result, melanoma lung metastasis development was inhibited. Endothelial homeostasis plays a key role in metastasis and enhancing the endothelial barrier integrity using the bioavailable S1PR1 agonist nanoliposomal formulation was a potentially effective strategy to reduce metastasis.

### REFERENCES CITED

1. Chaffer C L, Weinberg R A. A perspective on cancer cell metastasis. Science (New York, N.Y.) 2011; 331(6024):1559-64.
2. Guan X. Cancer metastases: challenges and opportunities. Acta Pharm Sin B 2015; 5(5):402-18.
3. Seyfried T N, Huysentruyt L C. On the origin of cancer metastasis. Crit Rev Oncog 2013; 18(1-2):43-73.
4. Zeeshan R, Mutahir Z. Cancer metastasis—tricks of the trade. Bosn J Basic Med Sci 2017; 17(3):172-82.
5. Poste G, Fidler I J. The pathogenesis of cancer metastasis. Nature 1980; 283(5743):139-46.
6. Reymond N, d'Agua B B, Ridley A J. Crossing the endothelial barrier during metastasis. Nature reviews Cancer 2013; 13(12):858-70.
7. Blazejczyk A, Papiernik D, Porshneva K, Sadowska J, Wietrzyk J. Endothelium and cancer metastasis: Perspectives for antimetastatic therapy. Pharmacol Rep 2015; 67(4):711-8.
8. Frolow M, Drozdz A, Kowalewska A, Nizankowski R, Chlopicki S. Comprehensive assessment of vascular health in patients; towards endothelium-guided therapy. Pharmacol Rep 2015; 67(4):786-92.
9. Walczak M, Suraj J, Kus K, Kij A, Zakrzewska A, Chlopicki S. Towards a comprehensive endothelial biomarkers profiling and endothelium-guided pharmacotherapy. Pharmacol Rep 2015; 67(4):771-7.
10. Garcia-Roman J, Zentella-Dehesa A. Vascular permeability changes involved in tumor metastasis. Cancer Lett 2013; 335(2):259-69.
11. Roblek M, Protsyuk D, Becker P F, Stefanescu C, Gorzelanny C, Glaus Garzon J F, et al. CCL2 Is a Vascular Permeability Factor Inducing CCR2-Dependent Endothelial Retraction during Lung Metastasis. Mol Cancer Res 2019; 17(3):783-93.
12. Weidert E, Pohler S E, Gomez E W, Dong C. Actinomyosin contraction, phosphorylation of VE-cadherin, and actin remodeling enable melanoma-induced endothelial cell-cell junction disassembly. PloS one 2014; 9(9):e108092.
13. Yurkovetsky Z R, Kirkwood J M, Edington H D, Marrangoni A M, Velikokhatnaya L, Winans M T, et al. Multiplex analysis of serum cytokines in melanoma patients treated with interferon-alpha2b. Clinical cancer research: an official journal of the American Association for Cancer Research 2007; 13(8):2422-8.
14. Zhang P, Feng S, Liu G, Wang H, Zhu H, Ren Q, et al. Mutant B-Raf(V600E) Promotes Melanoma Paracellular Transmigration by Inducing Thrombin-mediated Endothelial Junction Breakdown. The Journal of biological chemistry 2016; 291(5):2087-106.
15. Wallez Y, Huber P. Endothelial adherens and tight junctions in vascular homeostasis, inflammation and angiogenesis. Biochimica et biophysica acta 2008; 1778(3):794-809.
16. Vincent P A, Xiao K, Buckley K M, Kowalczyk A P. VE-cadherin: adhesion at arm's length. American journal of physiology Cell physiology 2004; 286(5):C987-97.
17. Rigor R R, Shen Q, Pivetti C D, Wu M H, Yuan S Y Myosin light chain kinase signaling in endothelial barrier dysfunction. Medicinal research reviews 2013; 33(5):911-33.
18. Cai S, Pestic-Dragovich L, O'Donnell M E, Wang N, Ingber D, Elson E, et al. Regulation of cytoskeletal mechanics and cell growth by myosin light chain phosphorylation. The American journal of physiology 1998; 275(5):C1349-56.
19. Garcia J G, Davis H W, Patterson C E. Regulation of endothelial cell gap formation and barrier dysfunction: role of myosin light chain phosphorylation. Journal of cellular physiology 1995; 163(3):510-22.
20. Winkler M S, Nierhaus A, Poppe A, Greiwe G, Graler M H, Daum G. Sphingosine-1-Phosphate: A Potential Biomarker and Therapeutic Target for Endothelial Dysfunction and Sepsis? Shock (Augusta, Ga.) 2017; 47(6):666-72.
21. Xiong Y, Hla T. S1P control of endothelial integrity. Curr Top Microbiol Immunol 2014; 378:85-105.
22. Zhang G, Xu S, Qian Y, He P. Sphingosine-1-phosphate prevents permeability increases via activation of endothelial sphingosine-1-phosphate receptor 1 in rat venules. American journal of physiology Heart and circulatory physiology 2010; 299(5):H1494-504.
23. Park S J, Im D S. Sphingosine 1-Phosphate Receptor Modulators and Drug Discovery. Biomolecules & therapeutics 2017; 25(1):80-90.
24. Sharma S, Mathur A G, Pradhan S, Singh D B, Gupta S. Fingolimod (FTY720): First approved oral therapy for multiple sclerosis. J Pharmacol Pharmacother 2011; 2(1):49-51.
25. Dyckman A J. Modulators of Sphingosine-1-phosphate Pathway Biology: Recent Advances of Sphingosine-1-phosphate Receptor 1 (S1P1) Agonists and Future Perspectives. Journal of medicinal chemistry 2017; 60(13):5267-89.
26. Pulla V K, Sriram D S, Soni V, Viswanadha S, Sriram D, Yogeeswari P. Targeting NAMPT for Therapeutic Intervention in Cancer and Inflammation: Structure-Based Drug Design and Biological Screening. Chem Biol Drug Des 2015; 86(4):881-94.
27. Shirvanyants D, Alexandrova A N, Dokholyan N V. Rigid substructure search. Bioinformatics 2011; 27(9):1327-9.
28. Gowda R, Dinavahi S S, Iyer S, Banerjee S, Neves R I, Pameijer C R, et al. Nanoliposomal delivery of cytosolic phospholipase A(2) inhibitor arachidonyl trimethyl ketone for melanoma treatment. Nanomedicine 2018; 14(3):863-73.
29. Dinavahi, S. S., Gowda, R., Gowda, K., C. G. Bazewicz, V. R. Chirasani, M. B. Battu, et al. Development of a novel multi-isoform ALDH inhibitor effective as an antimelanoma agent. Mol. Cancer Therapeut. 19(2) (2020), 447.
30. P. M. Reeves, Y-L. Kang, T. Kirchhausen, Endocytosis of ligand-activated sphingosine 1-phosphate receptor 1 mediated by the clathrin-pathway, Traffic 19 (1) (2016), 40-52.
31. Kendall S, Michel C C. The measurement of permeability in single rat venules using the red cell microperfusion technique. Experimental physiology 1995; 80(3):359-72.
32. Curry F E, Huxley V H, Sarelius I H. Techniques in the microcirculation: measure of permeability, pressure and flow. County Clare, Ireland; New York: Elsevier Scientific Publishers Ireland; Sole distributors for the U.S.A. and Canada, Elsevier Scientific Pub. Co.; 1983.
33. He P, Zhang X, Curry F E. Ca2+ entry through conductive pathway modulates receptormediated increase in microvessel permeability. The American journal of physiology 1996; 271(6 Pt 2):H2377-87.
34. Hale J J, Lynch C L, Neway W, Mills S G, Hajdu R, Keohane C A, et al. A rational utilization of high-throughput screening affords selective, orally bioavailable 1-benzyl-3-carboxyazetidine sphingosine-1-phosphate-1 receptor agonists. Journal of medicinal chemistry 2004; 47(27):6662-5.
35. Tanaka M, Ohshima T, Mitsuhashi H, Maruno M, Wakamatsu T. Total syntheses of the lignans isolated from Schisandra Chinensis. Tetrahedron 1995; 51(43):11693-702.
36. P. B. van Loenen, C. de Graaf, D. Verzijl, R. Leurs, D. Rognan, S. L. Peters, et al., Agonist-dependent effects of mutations in the sphingosine-1-phosphate type 1 receptor, Eur. J. Pharmacol. 667 (1-3) (2011) 105-112.
37. Ding F, Yin S, Dokholyan N V. Rapid flexible docking using a stochastic rotamer library of ligands. J Chem Inf Model 2010; 50(9):1623-32.
38. Minnear F L, Zhu L, He P. Sphingosine 1-phosphate prevents platelet-activating factorinduced increase in hydraulic conductivity in rat mesenteric venules: pertussis toxin sensitive. American journal of physiology Heart and circulatory physiology 2005; 289(2):H840-4.
39. Azzi S, Hebda J, GAVARD J. Vascular Permeability and Drug Delivery in Cancers.

Frontiers in Oncology 2013; 3(211).
40. Giannotta M, Trani M, Dejana E. VE-cadherin and endothelial adherens junctions: active guardians of vascular integrity. Developmental cell 2013; 26(5):441-54.
41. Mehta D, Malik A B. Signaling mechanisms regulating endothelial permeability.

Physiological reviews 2006; 86(1):279-367.
42. Petreaca M L, Yao M, Liu Y, Defea K, Martins-Green M. Transactivation of vascular endothelial growth factor receptor-2 by interleukin-8 (IL-8/CXCL8) is required for IL-8/CXCL8-induced endothelial permeability. Mol Biol Cell 2007; 18(12):5014-23.
40. Mehta D, Konstantoulaki M, Ahmmed G U, Malik A B. Sphingosine 1-phosphate-induced mobilization of intracellular Ca2+ mediates rac activation and adherens junction assembly in endothelial cells. The Journal of biological chemistry 2005; 280(17):17320-8.
41. Komarova Y A, Mehta D, Malik A B. Dual regulation of endothelial junctional permeability. Science's STKE: signal transduction knowledge environment 2007; 2007(412):re8.
42. Sanna M G, Liao J, Jo E, Alfonso C, Ahn M Y, Peterson M S, et al. Sphingosine 1-phosphate (S1P) receptor subtypes S1P1 and S1P3, respectively, regulate lymphocyte recirculation and heart rate. The Journal of biological chemistry 2004; 279(14):13839-48.
43. Din F U, Aman W, Ullah I, Qureshi O S, Mustapha O, Shafique S, et al. Effective use of nanocarriers as drug delivery systems for the treatment of selected tumors. International journal of nanomedicine 2017; 12:7291-309.
44. Deshpande P P, Biswas S, Torchilin V P. Current trends in the use of liposomes for tumor targeting. Nanomedicine (London, England) 2013; 8(9):1509-28.
45. Chen Y C, Gowda R, Newswanger R K, Leibich P, Fell B, Rosenberg G, et al. Targeting cholesterol transport in circulating melanoma cells to inhibit metastasis. Pigment Cell Melanoma Res 2017; 30(6):541-52.
46. Gowda R, Dinavahi S S, Iyer S, Banerjee S, Neves R I, Pameijer C R, et al. Nanoliposomal delivery of cytosolic phospholipase A2 inhibitor arachidonyl trimethyl ketone for melanoma treatment. Nanomedicine 2018; 14(3):863-73.
47. Dinavahi S S, Gowda R, Gowda K, Bazewicz C G, Chirasani V R, Battu M B, et al.

Development of a novel multi-isoform ALDH inhibitor effective as an anti-melanoma agent.

Molecular cancer therapeutics 2019.
48. Gowda R, Kardos G, Sharma A, Singh S, Robertson G P. Nanoparticle-Based Celecoxib and Plumbagin for the Synergistic Treatment of Melanoma. Molecular cancer therapeutics 2017; 16(3):440-52.
49. Huh S J, Liang S, Sharma A, Dong C, Robertson G P. Transiently entrapped circulating tumor cells interact with neutrophils to facilitate lung metastasis development. Cancer research 2010; 70(14):6071-82.
50. D. Mehta, M. Konstantoulaki, G. U. Ahmmed, A. B. Malik, Sphingosine 1-phosphate-induced mobilization of intracellular Ca2+ mediates rac activation and adherens junction assembly in endothelial cells, J. Biol. Chem. 280 (17) (2005) 17320-17328.
51. YA. Komarova, D. Mehta, A. B. Malik, Dual regulation of endothelial junctional permeability, Sci. STKE: Signal Transduct. Knowl. Environ. 2007 (412) (2007).
52. M. G. Sanna, J. Liao, E. Jo, C. Alfonso, M. Y Ahn, M. S. Peterson, et al., Sphingosine 1-phosphate (S1P) receptor subtypes S1P1 and S1P3, respectively, regulate lymphocyte recirculation and heart rate, J. Biol. Chem. 279 (14) (2004) 13839-13848.
53. T. Takasaki, K. Hagihara, R. Satoh, R. Sugiura, More than just an immunosuppressant: the emerging role of FTY720 as a novel inducer of ROS and apoptosis, Oxidative medicine and cellular longevity 2018 (2018) 4397159.
54. V. Albinet, M. L. Bats, A. Huwiler, P. Rochaix, C. Chevreau, B. Segui, et al., Dual role of sphingosine kinase-1 in promoting the differentiation of dermal fibroblasts and the dissemination of melanoma cells, Oncogene 33 (26) (2014) 3364-3373.
55. E.-S. Kim, J.-S. Kim, S. G. Kim, S. Hwang, C. H. Lee, A. Moon, Sphingosine 1-phos-phate regulates matrix metalloproteinase-9 expression and breast cell invasion through S1P3-Gαq coupling, J. Cell Sci. 124 (13) (2011) 2220-2230.
56. Y Zeng, X. Yao, L. Chen, Z. Yan, J. Liu, Y Zhang, et al., Sphingosine-1-phosphate induced epithelial-mesenchymal transition of hepatocellular carcinoma via an MMP-7/syndecan-1/TGF-0 autocrine loop, Oncotarget 7 (39) (2016) 63324-63337.
57. C. M. Yoon, B. S. Hong, H. G. Moon, S. Lim, P.-G. Suh, Y-K. Kim, et al., Sphingosine-1-phosphate promotes lymphangiogenesis by stimulating S1P1/Gi/PLC/Ca2+ signaling pathways, Blood 112 (4) (2008) 1129-1138.
58. M. Yu, P. He, Y Liu, Y He, Y Du, M. Wu, et al., Hyaluroan-regulated lymphatic permeability through S1P receptors is crucial for cancer metastasis, Med. Oncol. 32 (1) (2014) 381.
59. S. Tohme, R. L. Simmons, A. Tsung, Surgery for cancer: a trigger for metastases, Canc. Res. 77 (7) (2017) 1548.

A number of embodiments of the disclosure have been described. Nevertheless, it will be understood that various modifications may be made without departing from the spirit and scope of the invention. Accordingly, other embodiments are within the scope of the following claims.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. Compound I of the formula: 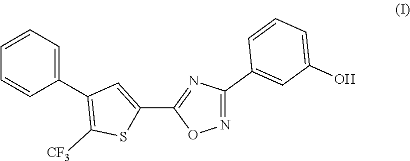 [SMILES: Oc1cccc(-c2noc(-c3cc(-c4ccccc4)c(C(F)(F)F)s3)n2)c1]
or a pharmaceutically acceptable salt, prodrug, or derivative thereof.

2. A pharmaceutical composition comprising the compound of claim 1, or a pharmaceutically acceptable salt, prodrug, or derivative thereof, and a pharmaceutically acceptable carrier.

3. A formulation comprising:
a plurality of nanoliposomes comprising a phospholipid and a PEG-phospholipid; and
the compound of claim 1, or a pharmaceutically acceptable salt, prodrug, or derivative thereof, encapsulated within the plurality of nanoliposomes.

4. The formulation of claim 3, wherein the phospholipid comprises L-α-phosphatidylcholine (ePC).

5. The formulation of claim 3, wherein the PEG-phospholipid comprises 1,2-dipalmitoyl-sn-glycero-3-phosphoethanolamine-N-[methoxy(polyethylene glycol)-2000] ammonium salt (DPPE-PEG 2000).

6. The formulation of claim 3, wherein the phospholipid and the PEG-phospholipid are present in a molar percent ratio ranging from about 50:50 to about 95:5.

7. The formulation of claim 6, wherein the phospholipid and the PEG-phospholipid are present in a molar percent ratio of about 80:20.

8. The formulation of claim 3, wherein the plurality of nanoliposomes have an average diameter ranging from about 50 nm to about 90 nm.

9. The formulation of claim 8, wherein the average diameter ranges from about 60 nm to about 80 nm.

10. A method of decreasing vascular permeability in a subject in need thereof, comprising administering a therapeutically effective amount of a compound of claim 1.

11. A method of treating diabetes or a cancer in a subject in need thereof, comprising administering a therapeutically effective amount of a compound of claim 1.

12. A method of preventing or inhibiting metastasis of a cancer in a subject in need thereof, comprising administering a therapeutically effective amount of a compound of claim 1.

13. The method of claim 12, wherein the cancer is selected from B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers, small cell lung cancer, non-small cell lung cancer, neuroblastoma, glioblastoma, ovarian cancer, pancreatic cancer, prostate cancer, skin cancers, melanoma, basal cell carcinoma, squamous cell carcinoma, liver cancer, squamous cell carcinomas of the mouth, throat, larynx, and lung, cervical cancer, cervical carcinoma, breast cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, AIDS-related lymphomas, or AIDS-related sarcomas.

14. The formulation of claim 9, wherein the average diameter ranges from about 65 nm to about 75 nm.
